(19) United States
(12) Patent Application Publication
(10) Pub. No.: US 2025/0295614 A1
(43) Pub. Date: Sep. 25, 2025

(54) COMPOSITIONS FOR PREVENTING TRANSITION FROM ACUTE TO CHRONIC PAIN

(71) Applicant: THE REGENTS OF THE UNIVERSITY OF CALIFORNIA, Oakland, CA (US)

(72) Inventors: Daniele PIOMELLI, Irvine, CA (US); Alex MABOU TAGNE, Irvine, CA (US); Yannick FOTIO, Irvine, CA (US)

(21) Appl. No.: 19/067,308

(22) Filed: Feb. 28, 2025

Related U.S. Application Data
(63) Continuation of application No. PCT/US2023/073788, filed on Sep. 8, 2023.
(60) Provisional application No. 63/375,113, filed on Sep. 9, 2022.

(51) Int. Cl. A61K 31/164; A61K 31/198; A61K 31/201; A61K 31/205; A61K 31/27; A61K 31/385; A61K 31/401; A61K 31/405; A61K 31/575; A61K 45/06; A61P 29/00

(52) U.S. Cl. CPC A61K 31/164; A61K 31/198; A61K 31/201; A61K 31/205; A61K 31/27; A61K 31/385; A61K 31/401; A61K 31/405; A61K 31/575; A61K 45/06; A61P 29/00

(57) ABSTRACT

Disclosed herein, inter alia, are methods of treating pain by administering compositions that include at least one purified free amino acid, at least one purified fatty acid, and a purified fatty acid amide, related compositions, and kits thereof.

### RELATED APPLICATIONS

This application is a continuation of and claims priority to the International Patent Application No. PCT/US2023/073788, filed Sep. 8, 2023, which claims the benefit of U.S. Provisional Application No. 63/375,113. filed Sep. 9, 2022, the contents of each of which are incorporated herein in their entireties.

### BACKGROUND

More than 1.5 billion people worldwide suffer from chronic pain, which often starts after an acute pain episode. The sequence of molecular events that lead to pain chronicity is still largely unknown but filling this gap is necessary to identify control nodes that might be targeted by preventive or disease-modifying therapies. Disclosed herein, inter alia, are solutions to these and other problems in the art.

### SUMMARY

In an aspect is provided a method of treating pain in a subject in need thereof, the method including administering an effective amount of a composition to said patient, said composition comprises at least one purified free amino acid, at least one purified fatty acid, and a purified fatty acid amide. In aspects that composition is administered orally. In aspects that purified fatty acid is oleic acid or erucic acid. In aspects the purified free amino acid is alanine, threonine, proline, serine, leucine, isoleucine, valine, phenylalanine, tyrosine, methionine, cysteine, or tryptophan.

In an aspect is provided a method of preventing chronic pain in a subject with acute pain, the method including administering an effective amount of a composition to said patient, said composition comprising at least one purified free amino acid, at least one purified fatty acid, and a purified fatty acid amide.

In an aspect is provided a method of preventing peripheral painful neuropathy in a subject previously treated with an anti-cancer agent, the method including administering an effective amount of a composition to said patient, said composition comprising at least one purified free amino acid, at least one purified fatty acid, and a purified fatty acid amide.

In an aspect is provided a method of preventing chronic pain in a subject with diabetes, the method including administering an effective amount of a composition to said patient, said composition comprises at least one purified free amino acid, at least one purified fatty acid, and a purified fatty acid amide.

In an aspect is provided a method of decreasing pain hypersensitivity in a subject after a traumatic pain event, the method comprising administering an effective amount of a composition to the subject, wherein the composition comprises at least one purified free amino acid, at least one purified fatty acid, and a purified fatty acid amide, wherein pain hypersensitivity is decreased in the subject.

In an aspect is provided a composition comprising at least one purified free amino acid, at least one purified fatty acid, and purified fatty acid amide.

In an aspect the methods above further include administering an effective amount of an agent to the subject, wherein said agent is a N-acylethanolamine Acid Amidase NAAA inhibitor, fatty acid amide hydrolase FAAH inhibitor, PPARα agonist, acetyl-L-carnitine, α-lipoic acid, or olesoxime.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1I. Dietary intervention prevents formalin-evoked acute nociception, long-lasting sensory abnormalities, emotional disturbances, and memory deficits. Mice were randomly assigned to receive either standard rodent chow (SD) or experimental diets (EDs: ED-1, ED-2 or COMBO) for a period of 3 weeks. Then, the animals were challenged with formalin (1% vol, intraplantar injection) or saline injections (Sham) and kept to their respective diets for a week. Nociceptive behaviors, paw edema, cognitive deficits (novel object recognition test, NOR) and emotional disturbances (elevated plus maze test, EPM) produced by formalin injections were evaluated at various time points post-injections (post-formalin day, PFD). To assess whether the dietary intervention permanently prevents the long-lasting effects of formalin, EDs were withdrawn and replaced by SD on PFD 7. (FIG. 1A) Time-course of the acute nocifensive response to 1% formalin. (FIG. 1B) Cumulative score of the phase 1 (Phase I: 0-10 min) and phase 2 (Phase II: 15-60 min) of the acute nocifensive response. (FIG. 1C) Paw edema (injected paw thickness minus non injected, in mm). (FIG. 1D-E) Mechanical and (FIGS. 1F-G) heat sensitivities to both ipsilateral (FIG. 1D, F) and contralateral (FIG. 1E, G) paws. (FIG. 1H) Anxiety-like behaviors in mice on PFD 7 assessed with EPM. Left, time in open arms (in seconds); right, anxiety index. (FIG. 1I) Long-term memory in mice that were subjected to sham or formalin, measured as discriminatory index on PFD 21. Results are expressed as mean±SEM (n=4-8, each group).

FIGS. 2A-2C Dietary intervention prevents spared nerve injury (SNI)-evoked long-lasting sensory hypersensitivities and memory deficit. Mice were randomly assigned to receive either standard rodent chow (SD) or experimental diets (EDs: ED-1, ED-2 or COMBO) for a period of 3 weeks. Then, the animals were subjected to sham or spared nerve injury (SNI) of their sciatic nerve and kept to their respective diets for two weeks. Nociceptive behaviors and cognitive disturbances (novel object recognition test, NOR) caused by SNI were evaluated on various time points post-surgery (post-SNI). To assess whether the dietary intervention permanently prevents the long-lasting effects of SNI, EDs were withdrawn and replaced by SD on post-SNI 14. (FIG. 2A) Mechanical allodynia in the operated (ipsilateral) limb. (FIG. 2B) Heat hyperalgesia in the ipsilateral paw. The contralateral limb was used as a control. (FIG. 2C) Long-term memory in mice that were subjected to sham or SNI and received either SD or EDs, measured as discriminatory index on post-SNI 24. Results are expressed as mean±SEM (n=7-8, each group).

FIGS. 3A-3C shows the changes in the levels of various spinal cord (L4-L6) metabolites in mice fed with standard diet (Form SD) or medical food (Form MF) 72 hours after intraplantar formalin administration; intermediates of glycolysis (FIG. 3A), intermediates of tricarboxylic acid (TCA) cycle (FIG. 3B), and purine derivatives including ATP (FIG. 3C). The medical food countered the shift from TCA to glycolysis and the energy crisis (low ATP levels) produced by formalin injection.

FIGS. 4A-4C shows the changes in the levels of various spinal cord (L4-L6) metabolites in mice fed with standard diet (Form SD) or medical food (Form MF) 72 hours after intraplantar formalin administration; amino acids (FIG. 4A), urea cycle intermediates (FIG. 4B), and acylated amino acids (FIG. 4C).

FIGS. 5A-5B shows the changes in the levels of various spinal cord (L4-L6) metabolites in mice fed with standard diet (Form SD) or medical food (Form MF) 72 hours after intraplantar formalin administration; fatty acids (FIG. 5A) and phospholipids (FIG. 5B).

FIG. 6 show plots of effects of the medical food on paw withdrawal latency after administration of interleukin-6 (IL-6, 5 ng, intraplantar) in male mice fed a standard diet (SD) after 1, 6, and 24 hours. Data are expressed as mean+/−SEM, n=8. ***P<0.001; **** P<0.0001, two-way ANOVA.

FIGS. 7A-7B show plots of effects of the medical food on paw withdrawal latency after administration of prostaglandin E₂ (PGE₂, 100 ng, intraplantar) in male mice primed with IL-6 on day 7 or 14 after IL-6 administration at 1, 6, and 24 hours. Data are expressed as mean+/−SEM, n=8. **P<0.01; ***P<0.001; **** P<0.0001, two-way ANOVA. Ns, not significant.

FIG. 8A shows a plot of effects of the medical food on morphine-induced antinociception in male mice. Morphine (mg/kg, subcutaneous) was administered in animals fed a standard diet or the medical food. The response is presented as percentage of Maximum Possible Effect (MPE). Also included are median affective dose (ED₅₀) values to produce antinociception in the two groups of mice. FIG. 8B shows a plot of effects of the medical food on tolerance to the antinociceptive effects of morphine in male mice. The antinociceptive effects of morphine (15 mg/kg, subcutaneous) were assessed on day 1. Tolerance was induced by administering morphine (30 mg/kg) on days 2-6. Tolerance was assessed on day 7 injecting 15 mg/kg morphine.

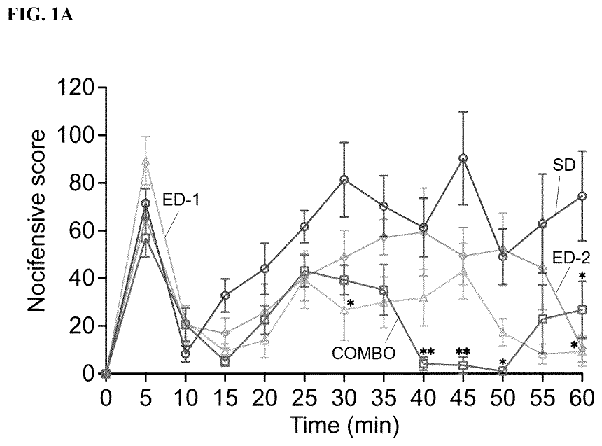

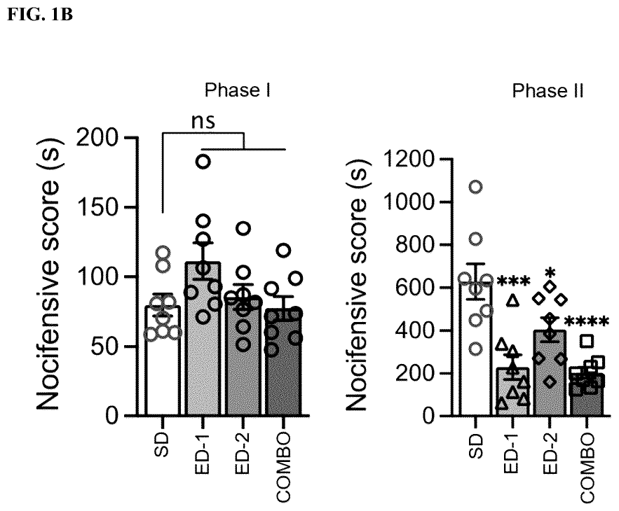

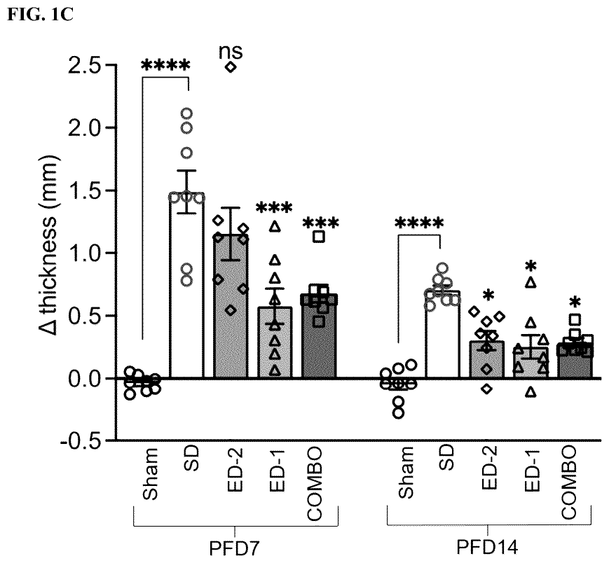

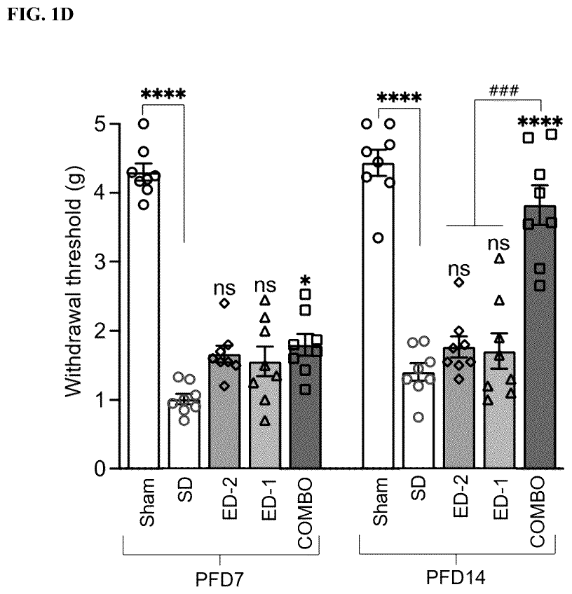

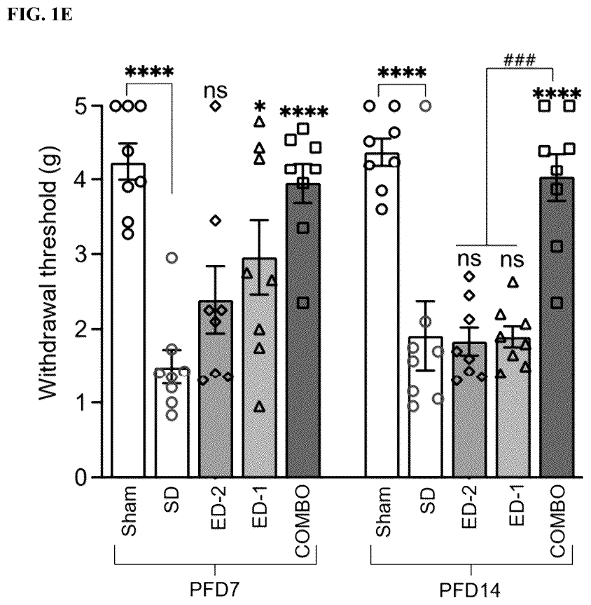

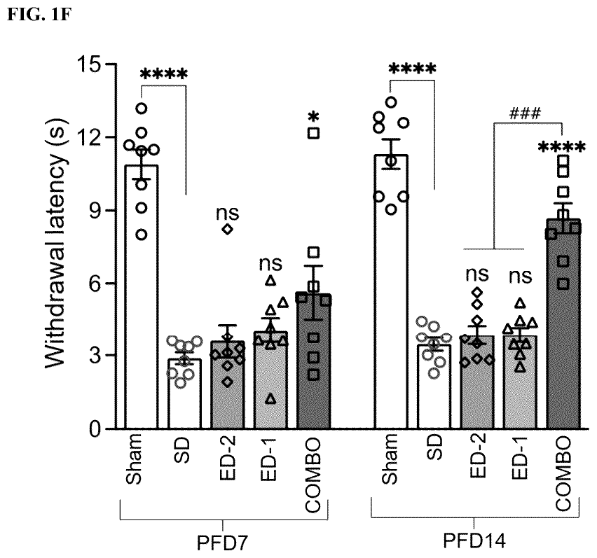

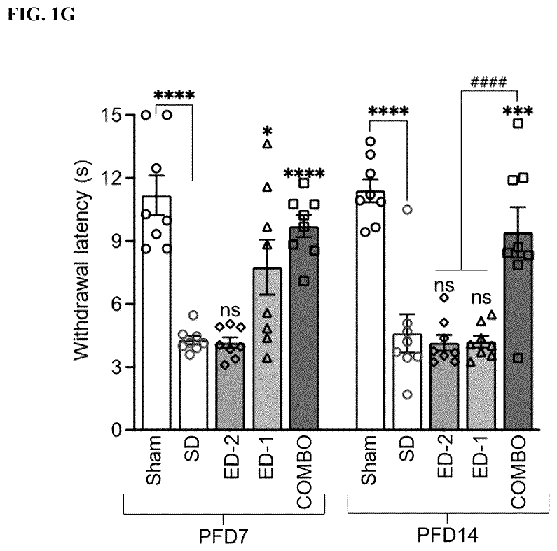

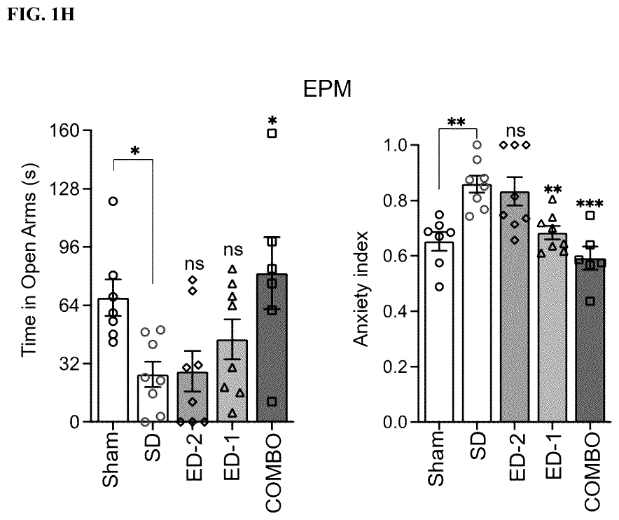

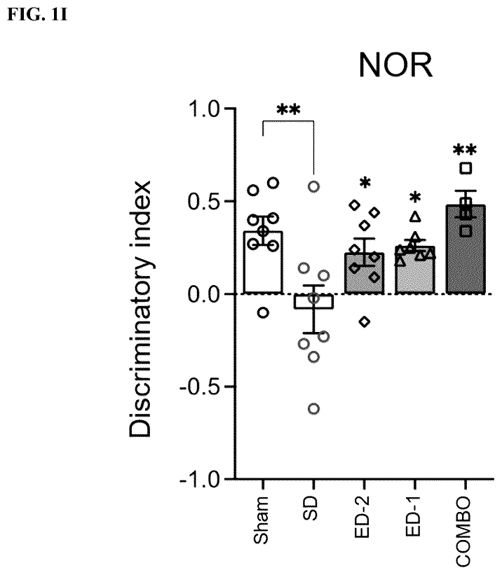

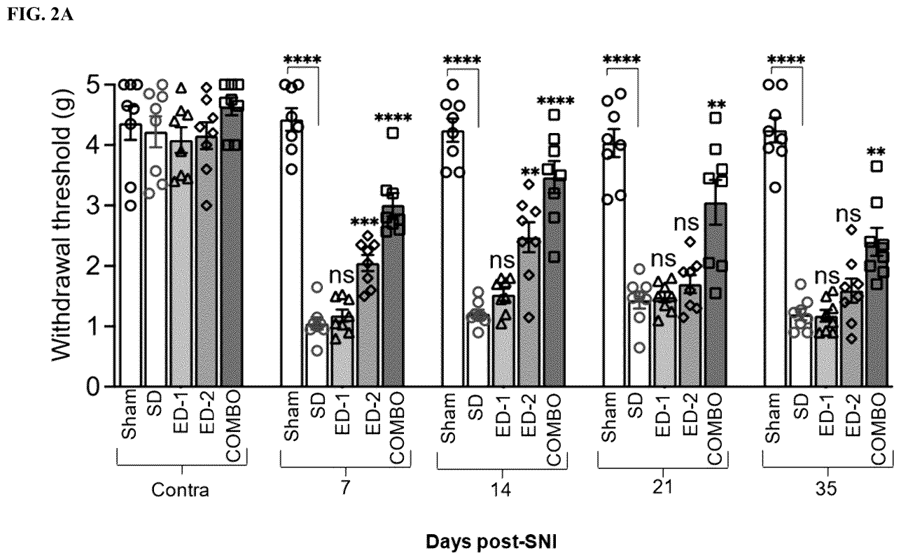

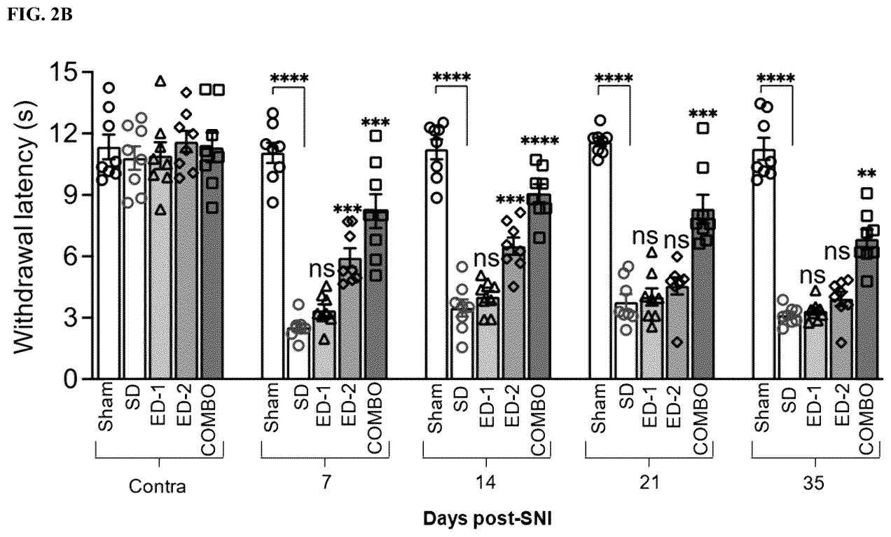

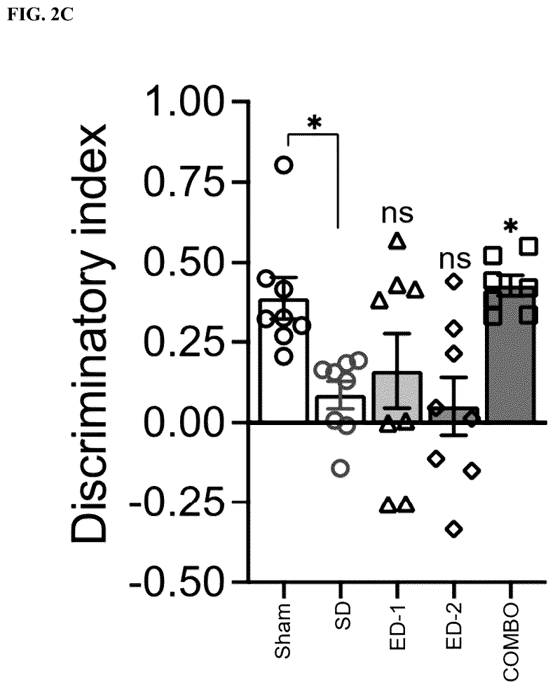

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (125I), or carbon-14 (14C). All isotopic variations of the compounds of the present disclosure, whether radioactive or not, are encompassed within the scope of the present disclosure.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C1-C20 alkyl, or unsubstituted 2 to 20 membered heteroalkyl,” the group may contain one or more unsubstituted C1-C20 alkyls, and/or one or more unsubstituted 2 to 20 membered heteroalkyls.

A “detectable agent” or “detectable moiety” is a substance (e.g., compound) or composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g., a first linker or second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like).

The term “capable of binding” as used herein refers to a moiety (e.g., a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waal's bond(s)/interactions, hydrogen bond(s), polar bond(s), or combinations or mixtures thereof).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. In embodiments, an amino acid can be a protein-bound amino acid (e.g., part of a peptide or protein) or a free amino acid (e.g., not part of peptide or protein). In embodiments, a free amino acid is exogenously administered.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “fatty acid” is used herein according to its plain ordinary meaning and refers to a lipid comprising a carboxylic acid with an aliphatic chain. In embodiments, the aliphatic chain is saturated or unsaturated. In embodiments, the aliphatic chain is unbranched. In embodiments, the aliphatic chain comprises a number of carbon atoms (e.g., 1, 2, 3, 4, 5, 6, 7, 8, 9, 10, etc.). In embodiments, the fatty acid is an ester. In embodiments, the ester is a triglyceride, phospholipid or cholesteryl ester. In embodiments, the fatty acid has biological activity (e.g., modulates a biological process).

The terms “palmitoylethanolamide” or “PEA” are used herein according to their plain ordinary meaning and refer any of the synthetic or naturally-occurring forms of the fatty acid amide palmitoylethanolamide, or variants or homologs thereof that maintain PEA activity (e.g., within at least 50%, 80%, 90%, 95%, 96%, 97%, 98%, 99% or 100% activity compared to PEA). In embodiments, the PEA is substantially identical to the compound identified by the CAS ID number 544-31-0 or a variant or homolog having substantial identity thereto. In embodiments, PEA targets peroxisome proliferator-activated receptor alpha (PPAR-α). In embodiments, PEA has affinity to G protein-coupled receptors (e.g., GPR55 and GPR119). In embodiments, PEA is an anti-inflammatory agent. In embodiments, PEA is an analgesic. In embodiments, PEA is an antimicrobial. In embodiments, PEA is an immunomodulatory agent. In embodiments, PEA has neuroprotective effects.

The terms “acetyl-L-carnitine”, “ALCAR” or “ALC” are used herein according to their plain ordinary meaning and refer to any of the synthetic or naturally-occurring forms of acetyl-L-carnitine or variants or homologs thereof that maintain acetyl-L-carnitine activity (e.g., within at least 50%, 80%, 90%, 95%, 96%, 97%, 98%, 99% or 100% activity compared to acetyl-L-carnitine). In embodiments, the acetyl-L-carnitine is substantially identical to the compound identified by the CAS ID number 14992-62-2 or a variant or homolog having substantial identity thereto.

The terms “alpha-lipoic acid” or “α-lipoic acid” are used herein according to their plain ordinary meaning and refer to any of the synthetic or naturally-occurring forms of α-lipoic acid, also known as thioctic acid, or variants or homologs thereof that maintain α-lipoic acid activity (e.g., within at least 50%, 80%, 90%, 95%, 96%, 97%, 98%, 99% or 100% activity compared to α-lipoic acid). In embodiments, the α-lipoic acid is substantially identical to the compound identified by the CAS ID number 1077-28-7 or a variant or homolog having substantial identity thereto.

The term “olesoxime” is used herein according to its plain ordinary meaning and refers to any of the forms of olesoxime, also known as TRO 19622, or variants or homologs thereof that maintain olesoxime activity (e.g., within at least 50%, 80%, 90%, 95%, 96%, 97%, 98%, 99% or 100% activity compared to olesoxime). In embodiments, the olesoxime is substantially identical to the compound identified by the CAS ID number 22033-87-0 or a variant or homolog having substantial identity thereto.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The terms “N-acylethanolamine-hydrolyzing acid amidase”, “N-acylethanolamine acid amide hydrolase” or “NAAA” are used herein according to their plain ordinary meaning and refer to any of the recombinant or naturally-occurring forms of N-acylethanolamine acid amide hydrolase or variants or homologs thereof that maintain NAAA activity (e.g., within at least 50%, 80%, 90%, 95%, 96%, 97%, 98%, 99% or 100% activity compared to NAAA). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring NAAA protein. In embodiments, the NAAA protein is substantially identical to the protein identified by the UniProt reference number Q02083 or a variant or homolog having substantial identity thereto.

The terms “fatty acid amide hydrolase” or “FAAH” are used herein according to their plain ordinary meaning and refer to any of the recombinant or naturally-occurring forms of fatty acid amide hydrolase or variants or homologs thereof that maintain FAAH activity (e.g., within at least 50%, 80%, 90%, 95%, 96%, 97%, 98%, 99% or 100% activity compared to FAAH). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring FAAH protein. In embodiments, the FAAH protein is substantially identical to the protein identified by the UniProt reference number 000519 or a variant or homolog having substantial identity thereto. In embodiments, the FAAH protein is substantially identical to the protein identified by the UniProt reference number Q6GMR7 or a variant or homolog having substantial identity thereto.

The terms “peroxisome proliferator-activated receptor alpha” or “PPARa” are used herein according to their plain ordinary meaning and refer to any of the recombinant or naturally-occurring forms of peroxisome proliferator-activated receptor alpha, also known as nuclear receptor subfamily 1, group C, member 1 (NR1C1), or variants or homologs thereof that maintain PPARα activity (e.g., within at least 50%, 80%, 90%, 95%, 96%, 97%, 98%, 99% or 100% activity compared to PPARa). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring PPARα protein. In embodiments, the PPARα protein is substantially identical to the protein identified by the UniProt reference number Q07869 or a variant or homolog having substantial identity thereto.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

An “inhibitor” refers to a compound (e.g., compounds described herein) that reduces activity when compared to a control, such as absence of the compound or a compound with known inactivity.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., a protein associated disease, a cancer (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

A “therapeutic agent” or “drug agent” as used herein refers to an agent (e.g., compound or composition) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being. A drug moiety is a monovalent drug. A therapeutic moiety is a monovalent therapeutic agent.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

As used herein, a “pathological state” refers to inflammatory conditions, neurodegenerative disorders, pain, a corneal neovascularization, diabetic retinopathy, dry macular degeneration, migraine, neuropathy, post herpetic neuralgia, trigeminal neuralgia, causalgia, diabetic neuropathy, chronic pain, nociceptive pain, complex regional pain syndrome (CRPS), neurogenic pain (including, but not limited to neuropathic pain, central pain and deafferentation pain), peripheral or polyneuropathic pain, toxic neuropathy, chronic neuropathy caused by chemotherapeutic and antiviral agents, nociceptive pain, or pruritus induced by uremia, pain associated with cancers, malignancies of various origin, polycythemia, jaundice or cholestasis, iron deficiency, athlete's foot, xerosis, wound healing, thyroid illness, hyperparathyroidism, or menopause, glossopharyngeal neuralgia, occipital neuralgia, pain, postherpetic neuralgia, retinopathy of prematurity, sinus headache, trigeminal neuralgia, or wet macular degeneration. In embodiments, pain, particularly severe pain, can be a stressor. In embodiments, provided herein are methods of treating chronic pain conditions, including neuropathic pain, and chronic or intermittent pain associated with chronic health conditions as such conditions are often substantial stressors.

In embodiments, “neuropathic pain” may include pain caused by a primary lesion or dysfunction of the nervous system. Such pain may be chronic and involve a maintained abnormal state of increased pain sensation, in which a reduction of pain threshold and the like are continued, due to persistent functional abnormalities ensuing from an injury or degeneration of a nerve, plexus or perineural soft tissue. Such injury or degeneration may be caused by wound, compression, infection, cancer, ischemia, or a metabolic or nutritional disorder such as diabetes mellitus. Neuropathic pain may include, but is not limited to, neuropathic allodynia wherein a pain sensation is induced by mechanical, thermal or another stimulus that does not normally provoke pain, neuropathic hyperalgesia wherein an excessive pain occurs in response to a stimulus that is normally less painful than experienced. Examples of neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. In embodiments, the neuropathic pain includes the pain caused by either central or peripheral nerve damage. In embodiments, it includes the pain caused by either mononeuropathy or polyneuropathy (e.g., familial amyloid polyneuropathy). In embodiments, as compared to inflammatory pain, neuropathic pain is resistant to therapy with nonsteroidal anti-inflammatory agents and opioid substances (e.g., morphine). Neuropathic pain may be bilateral in mirror image sites, or may be distributed approximately according to the innervation of the injured nerve, it may persist for months or years, and be experienced as burning, stabbing shooting, throbbing, piercing electric shock, or other unpleasant sensation.

The term “acute pain” is used in accordance with its plain and ordinary meaning and refers to the physiologic response and experience to noxious stimuli. In embodiments, the acute pain is sudden in onset and is time-limited. As used herein, the term “acute pain state” is a state of acute pain.

The term “chronic pain” is used in accordance with its plain and ordinary meaning and refers to pain that is ongoing and lasts longer than three months. In embodiments, the chronic pain lasts longer than six months. As used herein, the term “chronic pain state” is a state of chronic pain.

The term “pain hypersensitivity” is used interchangeably with “hyperalgesia” and “allodynia” and includes abnormally increased sensitivity to normally painful stimuli (hyperalgesia), and sensitivity to normally non-painful stimuli (allodynia). Opioid-induced hyperalgesia may develop as a result of long-term opioid use in the treatment of chronic pain.

As used herein, the term “inflammatory condition” refers to a disease or condition characterized by aberrant inflammation (e.g., an increased level of inflammation compared to a control such as a healthy person not suffering from a disease). Examples of inflammatory condition include postoperative cognitive dysfunction, traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barré syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning and includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. In embodiments, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

As a non-limiting example, the compounds described herein can be co-administered with anti-cancer agents or conventional chemotherapeutic agents including alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, methotrexate, leucovorin, capecitabine, cytarabine, floxuridine, fludarabine, gemcitabine, pemetrexed, raltitrexed, etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin, etc.), and the like.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer, or with adjunctive agents that may not be effective alone, but may contribute to the efficacy of the active agent.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. In embodiments, an anti-cancer agent is an inhibitor of K-Ras, RAF, MEK, Erk, PI3K, Akt, RTK, or mTOR. In embodiments, an anti-cancer agent is an MDM2 inhibitor or a genotoxic anti-cancer agent. In embodiments, an anti-cancer agent is nutlin-1, nutlin-2, nutlin-3, nutlin-3a, nutlin-3b, YH239-EE, MI-219, MI-773, MI-77301, MI-888, MX69, RG7112, RG7388, RITA, idasanutlin, DS-3032b, or AMG232. In embodiments, an anti-cancer agent is an alkylating agent, intercalating agent, or DNA replication inhibitor. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-la; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol.™ (i.e., paclitaxel), Taxotere.™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™) lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

“Chemotherapeutic” or “chemotherapeutic agent” is used in accordance with its plain ordinary meaning and refers to a chemical composition or compound having antineoplastic properties or the ability to inhibit the growth or proliferation of cells.

“Anti-diabetic agent” or “antidiabetic agent” is used in accordance with its plain ordinary meaning and refers to a composition (e.g., compound, drug, antagonist, inhibitor, modulator) having the ability to lower blood glucose levels in a subject. In some embodiments, an anti-diabetic agent is an agent identified herein having utility in methods of treating diabetes. In some embodiments, an anti-diabetic agent is an agent approved by the FDA or similar regulatory agency of a country other than the USA, for treating diabetes. Examples of anti-diabetic agents include, but are not limited to, insulin, insulin sensitizers (e.g., biguanides (e.g., metformin, phenformin, or buformin), thiazolidinediones (e.g., rosiglitazone, pioglitazone, troglitazone)), secretagogues (e.g., sulfonylureas (e.g., tolbutamide, acetohexamide, tolazamide, chlorpropamide, glipizide, glyburide, glibenclamide, glimepiride, gliclazide, glycopyramide, gliquidone), meglitinides (e.g., repaglinide, nateglinide)), alpha-glucosidase inhibitors (e.g., miglitol, acarbose, voglibose), peptide analog antidiabetic agents (e.g., incretins (glucagon-like peptide-1, gastric inhibitory peptide), glucagon-like peptide agonists (e.g., exenatide, liraglutide, taspoglutide), gastric inhibitoty peptide analogs, or dipeptidyl peptidase-4 inhibitors (e.g., vildagliptin, sitagliptin, saxagliptin, linagliptin, allogliptin, septagliptin), amylin agonist analogues (e.g., pramlintide).

The terms “N-acylethanolamine acid amidase”, “NAAA”, and “hNAAA” are used according to the plain and ordinary meaning in the art and refer to a 31 kDa enzyme by the same name involved in the hydrolysis of non-peptidic amides. The term “NAAA” may refer to the nucleotide sequence or protein sequence of human NAAA (e.g., Entrez 27163, Uniprot Q02083, RefSeq NM_014435, or RefSeq NP_055250). The term “NAAA” includes both the wild-type form of the nucleotide sequences or proteins as well as any mutants thereof. In some embodiments, “NAAA” is wild-type NAAA receptor. In some embodiments, “NAAA” is one or more mutant forms. The term “NAAA” XYZ refers to a nucleotide sequence or protein of a mutant NAAA wherein the Y numbered amino acid of NAAA that normally has an X amino acid in the wildtype, instead has a Z amino acid in the mutant. In embodiments, an NAAA is the human NAAA. In embodiments, the NAAA has the nucleotide sequence corresponding to reference number GI:109148549. In embodiments, the NAAA has the nucleotide sequence corresponding to RefSeq NM_014435.3. In embodiments, the NAAA has the protein sequence corresponding to reference number GI:109148550. In embodiments, the NAAA has the protein sequence corresponding to RefSeq NP_055250.2. In embodiments, NAAA functions in acidic conditions (e.g., pH about 4.5-5.0).

The term “FAAH” denotes a mammalian Fatty Acid Amide Hydrolase and includes, but is not limited to, the human, rat, and mouse forms of the enzyme. U.S. Pat. No. 6,271,015 discloses isolated and purified forms of FAAH. Fatty Amide Hydrolases (FAAHs) (Deutsch, D. G., et al., Prostaglandins Leukot. Essent. Fatty Acid, 66, 201-210 (2002)) are enzymes responsible for the degradation of lipid ethanolamides (Fowler, C. J., et al., Biochem. Pharmacol. 62, 517-526 (2001); Patricelli, M. P., et al., Vitam. Horm., 62, 663-674 (2001)), e.g., anandamide (Devane, W. A., et al., Science 258, 1946-1949 (1992)), oleoylethanolamide (Rodríguez de Fonseca, F., et al., Nature (London) 414, 209-212 (2001); Fu, J., et al., Nature (London) 425, 90-93 (2003)), and palmitoylethanolamide (Calignano, A., et al., Nature (London) 394, 277-281 (1998); Lambert, D. M., et al., Curr. Med. Chem. 9, 663-674 (2002)). Owing to the various and important physiological roles of fatty acid ethanolamides, classes of small-molecule compounds able to block FAAH or FAAHs but not bind to other endocannabinoid-metabolizing enzymes, e.g., monoglyceride lipase (MGL) (Dinh, T. P., et al., Proc. Natl. Acad. Sci. U.S.A. 99, 10819-10824 (2002)), or cannabinoid receptors, would be advantageous both as pharmacological tools and as prototypes for drug development projects (Piomelli, D., et al., Trends Pharmacol. Sci. 21, 218-224 (2000); Bisogno, T., et al., Curr. Pharm. Des. 8, 533-547 (2002); Yarnell, A., Chem. Eng. News 80(49), 32 (2002); Smith, A., Nat. Rev. Drug Discov. 2, 92 (2003); Wendeler, M., et al., Angew. Chem. Int. Ed. 42, 2938-2941 (2003)).

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

### II. Compositions

Provided herein are, inter alia, compositions for treating or preventing pain (e.g., treating) in a subject in need thereof, comprising at least one purified free amino acid, at least one purified fatty acid, and a purified fatty acid amide and/or pharmaceutically acceptable salts thereof.

In aspects the fatty acid amide is palmitoylethanolamide (PEA), oleoylethanolamide (OEA), erucylethanolamide, or stearoylethanolamide (SEA).

In aspects the purified fatty acid is oleic acid, palmitic acid, or erucic acid.

In aspects the purified free amino acid is alanine, threonine, proline, serine, leucine, isoleucine, valine, phenylalanine, tyrosine, methionine, cysteine, or tryptophan.

In aspects the purified free amino acid is L-arginine, L-asparagine, L-isoleucine, L-leucine, L-methionine, L-proline, L-serine, L-threonine, or L-tyrosine.

In aspects the purified free amino acid is ornithine, citrulline, or argininosuccinate.

In aspects the purified free amino acid is an acylated amino acid. In aspects the acetylated amino acid is an N-acetyl-L-phenylalanine, an N-acetylhistidine, an N-acetylthreonine, an N-acetylasparagine, an N-methyl-L-isoleucine, or an N-Acetylornithine.

In aspects, the compositions comprise at least two fatty acids. In aspects, the compositions comprise at least three fatty acids.

In aspects, the compositions comprise at least two purified free amino acids. In aspects, the compositions comprise at least three purified free amino acids. In aspects, the compositions comprise at least four purified free amino acids. In aspects, the compositions comprise at least five purified free amino acids. In aspects, the compositions comprise at least six purified free amino acids. In aspects, the compositions comprise at least seven purified free amino acids. In aspects, the compositions comprise at least eight purified free amino acids. In aspects, the compositions comprise at least nine purified free amino acids. In aspects, the compositions comprise at least 10 purified free amino acids. In aspects, the compositions comprise at least 11 purified free amino acids. In aspects, the compositions comprise at least 12 purified free amino acids.

In an aspect, the compositions have an amino acid/fatty acid amide ratio of 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 70:1, 80:1, 90:1, or 100:1 weight/weight (w/w).

In an aspect, the compositions have an fatty acid/fatty acid amide ratio of 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 70:1, 80:1, 90:1, or 100:1 weight/weight (w/w).

In an aspect, the compositions have an amino acid/fatty acid amide ratio of 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 70:1, 80:1, 90:1, or 100:1 weight/weight (w/w).

In an aspect, the compositions have an amino acid/fatty acid ratio of 1:1, 2:1, 3:1, 4:1, 5:1, 6:1, 7:1, 8:1, 9:1, 10:1, 15:1, or 20:1 weight/weight (w/w).

In an aspect compositions for treating pain in a subject in need thereof, comprising at least one purified free amino acid, at least one purified fatty acid, and a purified palmitoylethanolamide (PEA).

In an aspect compositions for preventing pain in a subject in need thereof, comprising at least one purified free amino acid, at least one purified fatty acid, and a purified palmitoylethanolamide (PEA).

In an aspect the compositions further include an agent, wherein said agent is a NAAA inhibitor, FAAH inhibitor, PPARα agonist, acetyl-L-carnitine, α-lipoic acid, or olesoxime.

In an aspect, the composition is a liquid. In an aspect the composition is a solid. In an aspect the composition is a powder. In an aspect, the powder can be dissolved in a liquid suitable for human consumption, such as water or a fruit juice. In an aspect the composition can be in a suitable form, such as a solid bar, as a paste, gel, tablet, capsule, or liquid.

In an aspect the compositions further includes a pharmaceutically acceptable excipient, such as binders (e.g., pre-gelatinized corn starch, polyvinylpyrrolidone, or hydroxypropyl methylcellulose); filling agents (e.g., lactose, microcrystalline cellulose or calcium hydrogen phosphate); lubricants (e.g., magnesium stearate, talc, or silica); disintegrating agents (e.g., potato starch or sodium starch glycolate); or inhibiting agents (e.g., sodium lauryl sulfate). In an aspect, the tablets can be coated by methods well known in the art. In an aspect, the liquid preparations for oral administration can be, for example, in the form of solutions, syrups or suspensions, or they can be in the form of lyophilized products to be reconstituted, before their use, with water or other suitable vehicles. Such liquid preparations can be prepared by conventional methods with pharmaceutically acceptable additives, such as suspending agents (e.g., sorbitol syrup, cellulose derivatives, or edible hydrogenated fats); emulsifying agents (e.g., lecithin or acacia); non-aqueous vehicles (e.g., almond oil, oily esters, ethyl alcohol, or fractionated plant-based oils); and preservatives (e.g., methyl- or propyl-p-hydroxybenzoates or sorbic acid).

In an aspect the composition further comprises a sweetening agent (e.g., sucrose, fructose, honey, or molasses) or sweeteners, for example artificial sweeteners (e.g., lactitol, maltitol, cyclamate, aspartame, isomalt, mannitol, sorbitol, xylitol, or erythritol).

In an aspect the composition further comprises a flavoring agent.

In an aspect a dosage form of the composition is provided. In an aspect, the dosage form is liquid or solid. In an aspect the dosage form is a drink or a syrup. In an aspect, the dosage form is a tablet, a capsule, or a sachet. In an aspect the dosage from comprises about 1 gram to about 150 grams of the composition. In an aspect the dosage form comprises about 1 gram to about 10 grams of the composition. In an aspect the dosage form comprises about 2 grams to about 9 grams of the composition. In an aspect the dosage form comprises about 3 grams to about 8 grams of the composition. In an aspect the dosage form comprises about 4 grams to about 7 grams of the composition. In an aspect the dosage form comprises about 5 grams to about 6 grams of the composition. In an aspect the dosage form comprises about 10 grams to about 20 grams of the composition. In an aspect the dosage form comprises about 20 grams to about 30 grams of the composition. In an aspect the dosage form comprises about 30 grams to about 40 grams of the composition. In an aspect the dosage form comprises about 40 grams to about 50 grams of the composition. In an aspect the dosage form comprises about 50 grams to about 60 grams of the composition. In an aspect the dosage form comprises about 60 grams to about 70 grams of the composition. In an aspect the dosage form comprises about 70 grams to about 80 grams of the composition. In an aspect the dosage form comprises about 80 grams to about 90 grams of the composition. In an aspect the dosage form comprises about 90 grams to about 100 grams of the composition. In an aspect the dosage form comprises about 100 grams to about 110 grams of the composition. In an aspect the dosage form comprises about 110 grams to about 120 grams of the composition. In an aspect the dosage form comprises about 120 grams to about 130 grams of the composition. In an aspect the dosage form comprises about 130 grams to about 140 grams of the composition. In an aspect the dosage form comprises about 140 grams to about 150 grams of the composition. In an aspect the dosage form comprises about 1 gram of the composition. In an aspect the dosage form comprises about 2 grams of the composition. In an aspect the dosage form comprises about 3 grams of the composition. In an aspect the dosage form comprises about 4 grams of the composition. In an aspect the dosage form comprises about 5 grams of the composition. In an aspect the dosage form comprises about 6 grams of the composition. In an aspect the dosage form comprises about 7 grams of the composition. In an aspect the dosage form comprises about 8 grams of the composition. In an aspect the dosage form comprises about 9 grams of the composition. In an aspect the dosage form comprises about 10 grams of the composition. In an aspect the dosage form comprises about 20 grams of the composition. In an aspect the dosage form comprises about 30 grams of the composition. In an aspect the dosage form comprises about 40 grams of the composition. In an aspect the dosage form comprises about 50 grams of the composition. In an aspect the dosage form comprises about 60 grams of the composition. In an aspect the dosage form comprises about 70 grams of the composition. In an aspect the dosage form comprises about 80 grams of the composition. In an aspect the dosage form comprises about 90 grams of the composition. In an aspect the dosage form comprises about 100 grams of the composition. In an aspect the dosage form comprises about 110 grams of the composition. In an aspect the dosage form comprises about 120 grams of the composition. In an aspect the dosage form comprises about 130 grams of the composition. In an aspect the dosage form comprises about 140 grams of the composition. In an aspect the dosage form comprises about 150 grams of the composition.

### III. Methods

Provided herein are, inter alia, methods for treating or preventing pain (e.g., treating) in a subject in need thereof, comprising administering a composition described herein to a patient in need thereof.

In an aspect is provided a method of treating pain in a subject in need thereof, the method including administering an effective amount of a composition to said patient, the composition comprising at least one purified free amino acid, at least one purified fatty acid, and a purified fatty acid amide.

In an aspect is provided a method of treating pain in a subject in need thereof, the method including administering an effective amount of a composition to said patient, the composition comprising at least one purified free amino acid, at least one purified fatty acid, and purified palmitoylethanolamide (PEA).

In an aspect is provided a method of preventing chronic pain in a subject in need thereof, the method including administering an effective amount of a composition to said patient, the composition comprising at least one purified free amino acid, at least one purified fatty acid, and a purified fatty acid amide.

In an aspect is provided a method of preventing chronic pain in a subject after a traumatic pain event, the method comprising administering an effective amount of a composition to the subject, wherein the composition comprises at least one purified free amino acid, at least one purified fatty acid, and purified palmitoylethanolamide (PEA), wherein chronic pain is prevented in the subject.

In an aspect is provided a method of preventing peripheral neuropathic pain in a subject with cancer, the method comprising administering an effective amount of a composition to the subject, wherein the composition comprises at least one purified free amino acid, at least one purified fatty acid, and a purified fatty acid amide, wherein chronic pain is prevented in the subject.

In an aspect is provided a method of preventing peripheral neuropathic pain in a subject with cancer, the method comprising administering an effective amount of a composition to the subject, wherein the composition comprises at least one purified free amino acid, at least one purified fatty acid, and purified palmitoylethanolamide (PEA), wherein chronic pain is prevented in the subject.

In an aspect is provided a method of preventing chronic pain in a subject with diabetes, the method comprising administering an effective amount of a composition to the subject, wherein the composition comprises at least one purified free amino acid, at least one purified fatty acid, and a purified fatty acid amide, wherein chronic pain is prevented in the subject.

In an aspect is provided a method of preventing chronic pain in a subject with diabetes, the method comprising administering an effective amount of a composition to the subject, wherein the composition comprises at least one purified free amino acid, at least one purified fatty acid, and purified palmitoylethanolamide (PEA), wherein chronic pain is prevented in the subject.

In an aspect is provided a method of decreasing pain hypersensitivity in a subject after a traumatic pain event, the method comprising administering an effective amount of a composition to the subject, wherein the composition comprises at least one purified free amino acid, at least one purified fatty acid, and a purified fatty acid amide, wherein pain hypersensitivity is decreased in the subject.

In an aspect is provided a method of decreasing pain hypersensitivity in a subject after a traumatic pain event, the method comprising administering an effective amount of a composition to the subject, wherein the composition comprises at least one purified free amino acid, at least one purified fatty acid, and purified palmitoylethanolamide (PEA), wherein pain hypersensitivity is decreased in the subject.

In aspects, the pain hypersensitivity is induced by acute/inflammatory pain. In aspects, the inflammatory pain is IL-6 mediated.

In aspects, the composition is administered orally. In aspects, the composition is provided as a supplement to food. In an aspect, the composition is administered without food. In aspects the oral form is selected from the group consisting of tablet, capsule, granule, powder, oily pearl, solution, suspension, and aerosol.

In aspects, the at least one purified fatty acid is oleic acid or erucic acid. In aspects, the composition includes oleic acid and erucic acid.

In aspects, the at least one purified free amino acid is alanine, threonine, proline, serine, leucine, isoleucine, valine, phenylalanine, tyrosine, methionine, cysteine, or tryptophan. In aspects, the compositions comprise at least two purified free amino acids. In aspects, the compositions comprise at least three purified free amino acids. In aspects, the compositions comprise at least four purified free amino acids. In aspects, the compositions comprise at least five purified free amino acids. In aspects, the compositions comprise at least six purified free amino acids. In aspects, the compositions comprise at least seven purified free amino acids. In aspects, the compositions comprise at least eight purified free amino acids. In aspects, the compositions comprise at least nine purified free amino acids. In aspects, the compositions comprise at least 10 purified free amino acids. In aspects, the compositions comprise at least 11 purified free amino acids. In aspects, the compositions comprise at least 12 purified free amino acids.

In aspects, the dosages for each one or more compound are provided in Table 1.

TABLE 1

| | Animal dose | Human equivalent |
|---|---|---|
| Compound | in mg/kg | dose in mg/kg |
| PEA | 103.75 | 8.3 |
| Oleic acid | 6200 | 496 |
| Erucic acid | 310 | 24.8 |
| Alanine | 2362.5 | 189 |
| Threonine | 1625 | 130 |
| Proline | 3000 | 240 |
| Serine | 1250 | 100 |
| Leucine | 3875 | 310 |
| Isoleucine | 3062.5 | 245 |
| Valine | 2875 | 230 |
| Phenylalanine | 1875 | 150 |
| Tyrosine | 1125 | 90 |
| Methionine | 750 | 60 |
| Cysteine | 562.5 | 45 |
| Tryptophan | 762.5 | 61 |

In aspects, the dosage ranges for each one or more compound are provided in Table 2.

TABLE 2

| | Animal | Human | Human |
|---|---|---|---|
| | dose range | equivalent dose | equivalent dose |
| Compound | in mg/kg ¹ | range in mg/kg | range in mg ² |
| PEA | 10-104 | 0.8-9 | 56-581 |
| Oleic acid | 600-6200 | 48-496 | 3360-34720 |
| Erucic acid | 30-310 | 2-25 | 168-1736 |
| Alanine | 250-2363 | 20-189 | 1400-13230 |
| Threonine | 160-1625 | 12-130 | 896-9100 |
| Proline | 300-3000 | 24-240 | 1680-16800 |
| Serine | 130-1250 | 10-100 | 728-7000 |
| Leucine | 350-3875 | 28-310 | 1960-21700 |
| Isoleucine | 300-3063 | 24-245 | 1680-17150 |
| Valine | 285-2875 | 22-230 | 1596-16100 |
| Phenylalanine | 190-1875 | 15-150 | 1064-10500 |
| Tyrosine | 120-1125 | 9-90 | 672-6300 |
| Methionine | 75-750 | 6-60 | 420-4200 |
| Cysteine | 50-563 | 4-45 | 280-3150 |
| Tryptophan | 78-763 | 6-61 | 434-4270 |

¹ Assume mice consume 5 g of chow per day
² Assume 70 kg human

In an aspect the dosage of PEA is about 0.8 mg/kg to about 9 mg/kg. In aspects, the dosage of PEA is about 0.9 mg/kg to about 9 mg/kg. In aspects, the dosage of PEA is about 1 mg/kg to about 9 mg/kg. In aspects, the dosage of PEA is about 2 mg/kg to about 9 mg/kg. In aspects, the dosage of PEA is about 3 mg/kg to about 9 mg/kg. In aspects, the dosage of PEA is about 4 mg/kg to about 9 mg/kg. In aspects, the dosage of PEA is about 5 mg/kg to about 9 mg/kg. In aspects, the dosage of PEA is about 6 mg/kg to about 9 mg/kg. In aspects, the dosage of PEA is about 7 mg/kg to about 9 mg/kg. In aspects, the dosage of PEA is about 8 mg/kg to about 9 mg/kg.

In aspects, the dosage of PEA is about 0.8 mg/kg to about 8 mg/kg. In aspects, the dosage of PEA is about 0.8 mg/kg to about 7 mg/kg. In aspects, the dosage of PEA is about 0.8 mg/kg to about 6 mg/kg. In aspects, the dosage of PEA is about 0.8 mg/kg to about 5 mg/kg. In aspects, the dosage of PEA is about 0.8 mg/kg to about 4 mg/kg. In aspects, the dosage of PEA is about 0.8 mg/kg to about 3 mg/kg. In aspects, the dosage of PEA is about 0.8 mg/kg to about 2 mg/kg. In aspects, the dosage of PEA is about 0.8 mg/kg to about 1 mg/kg. In aspects, the dosage of PEA is about 0.8 mg/kg to about 0.9 mg/kg.

In aspects, the dosage of PEA is 0.8 mg/kg to 9 mg/kg. In aspects, the dosage of PEA is 0.8 mg/kg to 9 mg/kg. In aspects, the dosage of PEA is 0.9 mg/kg to 9 mg/kg. In aspects, the dosage of PEA is 1 mg/kg to 9 mg/kg. In aspects, the dosage of PEA is 2 mg/kg to 9 mg/kg. In aspects, the dosage of PEA is 3 mg/kg to 9 mg/kg. In aspects, the dosage of PEA is 4 mg/kg to 9 mg/kg. In aspects, the dosage of PEA is 5 mg/kg to 9 mg/kg. In aspects, the dosage of PEA is 6 mg/kg to 9 mg/kg. In aspects, the dosage of PEA is 7 mg/kg to 9 mg/kg. In aspects, the dosage of PEA is 8 mg/kg to 9 mg/kg.

In aspects, the dosage of PEA is 0.8 mg/kg to 8 mg/kg. In aspects, the dosage of PEA is 0.8 mg/kg to about 7 mg/kg. In aspects, the dosage of PEA is 0.8 mg/kg to 6 mg/kg. In aspects, the dosage of PEA is 0.8 mg/kg to 5 mg/kg. In aspects, the dosage of PEA is 0.8 mg/kg to 4 mg/kg. In aspects, the dosage of PEA is 0.8 mg/kg to 3 mg/kg. In aspects, the dosage of PEA is 0.8 mg/kg to 2 mg/kg. In aspects, the dosage of PEA is 0.8 mg/kg to 1 mg/kg. In aspects, the dosage of PEA is 0.8 mg/kg to 0.9 mg/kg.

In an aspect the dosage of oleic acid is about 48 mg/kg to about 496 mg/kg. In aspects, the dosage of oleic acid is about 49 mg/kg to about 496 mg/kg. In aspects, the dosage of oleic acid is about 50 mg/kg to about 496 mg/kg. In aspects, the dosage of oleic acid is about 75 mg/kg to about 496 mg/kg. In aspects, the dosage of oleic acid is about 100 mg/kg to about 496 mg/kg. In aspects, the dosage of oleic acid is about 150 mg/kg to about 496 mg/kg. In aspects, the dosage of oleic acid is about 200 mg/kg to about 496 mg/kg. In aspects, the dosage of oleic acid is about 250 mg/kg to about 496 mg/kg. In aspects, the dosage of oleic acid is about 300 mg/kg to about 496 mg/kg. In aspects, the dosage of oleic acid is about 350 mg/kg to about 496 mg/kg. In aspects, the dosage of oleic acid is about 400 mg/kg to about 496 mg/kg. In aspects, the dosage of oleic acid is about 450 mg/kg to about 496 mg/kg. In aspects, the dosage of oleic acid is about 475 mg/kg to about 496 mg/kg. In aspects, the dosage of oleic acid is about 490 mg/kg to about 496 mg/kg. In aspects, the dosage of oleic acid is about 495 mg/kg to about 496 mg/kg.

In aspects, the dosage of oleic acid is about 48 mg/kg to about 495 mg/kg. In aspects, the dosage of oleic acid is about 48 mg/kg to about 490 mg/kg. In aspects, the dosage of oleic acid is about 48 mg/kg to about 475 mg/kg. In aspects, the dosage of oleic acid is about 48 mg/kg to about 450 mg/kg. In aspects, the dosage of oleic acid is about 48 mg/kg to about 400 mg/kg. In aspects, the dosage of oleic acid is about 48 mg/kg to about 350 mg/kg. In aspects, the dosage of oleic acid is about 48 mg/kg to about 300 mg/kg. In aspects, the dosage of oleic acid is about 48 mg/kg to about 250 mg/kg. In aspects, the dosage of oleic acid is about 48 mg/kg to about 200 mg/kg. In aspects, the dosage of oleic acid is about 48 mg/kg to about 150 mg/kg. In aspects, the dosage of oleic acid is about 48 mg/kg to about 100 mg/kg. In aspects, the dosage of oleic acid is about 48 mg/kg to about 75 mg/kg. In aspects, the dosage of oleic acid is about 48 mg/kg to about 50 mg/kg. In aspects, the dosage of oleic acid is about 48 mg/kg to about 49 mg/kg.

In aspects, the dosage of oleic acid is 48 mg/kg to 496 mg/kg. In aspects, the dosage of oleic acid is 49 mg/kg to 496 mg/kg. In aspects, the dosage of oleic acid is 50 mg/kg to 496 mg/kg. In aspects, the dosage of oleic acid is 75 mg/kg to 496 mg/kg. In aspects, the dosage of oleic acid is 100 mg/kg to 496 mg/kg. In aspects, the dosage of oleic acid is 150 mg/kg to 496 mg/kg. In aspects, the dosage of oleic acid is 200 mg/kg to 496 mg/kg. In aspects, the dosage of oleic acid is 250 mg/kg to 496 mg/kg. In aspects, the dosage of oleic acid is 300 mg/kg to 496 mg/kg. In aspects, the dosage of oleic acid is 350 mg/kg to 496 mg/kg. In aspects, the dosage of oleic acid is 400 mg/kg to 496 mg/kg. In aspects, the dosage of oleic acid is 450 mg/kg to 496 mg/kg. In aspects, the dosage of oleic acid is 475 mg/kg to 496 mg/kg. In aspects, the dosage of oleic acid is 490 mg/kg to 496 mg/kg. In aspects, the dosage of oleic acid is 495 mg/kg to 496 mg/kg.

In aspects, the dosage of oleic acid is 48 mg/kg to 495 mg/kg. In aspects, the dosage of oleic acid is 48 mg/kg to 490 mg/kg. In aspects, the dosage of oleic acid is 48 mg/kg to 475 mg/kg. In aspects, the dosage of oleic acid is 48 mg/kg to 450 mg/kg. In aspects, the dosage of oleic acid is 48 mg/kg to 400 mg/kg. In aspects, the dosage of oleic acid is 48 mg/kg to 350 mg/kg. In aspects, the dosage of oleic acid is 48 mg/kg to 300 mg/kg. In aspects, the dosage of oleic acid is 48 mg/kg to 250 mg/kg. In aspects, the dosage of oleic acid is 48 mg/kg to 200 mg/kg. In aspects, the dosage of oleic acid is 48 mg/kg to 150 mg/kg. In aspects, the dosage of oleic acid is 48 mg/kg to 100 mg/kg. In aspects, the dosage of oleic acid is 48 mg/kg to 75 mg/kg. In aspects, the dosage of oleic acid is 48 mg/kg to 50 mg/kg. In aspects, the dosage of oleic acid is 48 mg/kg to 49 mg/kg.

In an aspect the dosage of erucic acid is about 2 mg/kg to about 25 mg/kg. In aspects, the dosage of erucic acid is about 3 mg/kg to about 25 mg/kg. In aspects, the dosage of erucic acid is about 4 mg/kg to about 25 mg/kg. In aspects, the dosage of erucic acid is about 5 mg/kg to about 25 mg/kg. In aspects, the dosage of erucic acid is about 6 mg/kg to about 25 mg/kg. In aspects, the dosage of erucic acid is about 7 mg/kg to about 25 mg/kg. In aspects, the dosage of erucic acid is about 8 mg/kg to about 25 mg/kg. In aspects, the dosage of erucic acid is about 9 mg/kg to about 25 mg/kg. In aspects, the dosage of erucic acid is about 10 mg/kg to about 25 mg/kg. In aspects, the dosage of erucic acid is about 11 mg/kg to about 25 mg/kg. In aspects, the dosage of erucic acid is about 12 mg/kg to about 25 mg/kg. In aspects, the dosage of erucic acid is about 13 mg/kg to about 25 mg/kg. In aspects, the dosage of erucic acid is about 14 mg/kg to about 25 mg/kg. In aspects, the dosage of erucic acid is about 15 mg/kg to about 25 mg/kg. In aspects, the dosage of erucic acid is about 16 mg/kg to about 25 mg/kg. In aspects, the dosage of erucic acid is about 17 mg/kg to about 25 mg/kg. In aspects, the dosage of erucic acid is about 18 mg/kg to about 25 mg/kg. In aspects, the dosage of erucic acid is about 19 mg/kg to about 25 mg/kg. In aspects, the dosage of erucic acid is about 20 mg/kg to about 25 mg/kg. In aspects, the dosage of erucic acid is about 21 mg/kg to about 25 mg/kg. In aspects, the dosage of erucic acid is about 22 mg/kg to about 25 mg/kg. In aspects, the dosage of erucic acid is about 23 mg/kg to about 25 mg/kg. In aspects, the dosage of erucic acid is about 24 mg/kg to about 25 mg/kg.

In aspects, the dosage of erucic acid is about 2 mg/kg to about 24 mg/kg. In aspects, the dosage of erucic acid is about 2 mg/kg to about 23 mg/kg. In aspects, the dosage of erucic acid is about 2 mg/kg to about 22 mg/kg. In aspects, the dosage of erucic acid is about 2 mg/kg to about 21 mg/kg. In aspects, the dosage of erucic acid is about 2 mg/kg to about 20 mg/kg. In aspects, the dosage of erucic acid is about 2 mg/kg to about 19 mg/kg. In aspects, the dosage of erucic acid is about 2 mg/kg to about 18 mg/kg. In aspects, the dosage of erucic acid is about 2 mg/kg to about 17 mg/kg. In aspects, the dosage of erucic acid is about 2 mg/kg to about 16 mg/kg. In aspects, the dosage of erucic acid is about 2 mg/kg to about 15 mg/kg. In aspects, the dosage of erucic acid is about 2 mg/kg to about 14 mg/kg. In aspects, the dosage of erucic acid is about 2 mg/kg to about 13 mg/kg. In aspects, the dosage of erucic acid is about 2 mg/kg to about 12 mg/kg. In aspects, the dosage of erucic acid is about 2 mg/kg to about 11 mg/kg. In aspects, the dosage of erucic acid is about 2 mg/kg to about 10 mg/kg. In aspects, the dosage of erucic acid is about 2 mg/kg to about 9 mg/kg. In aspects, the dosage of erucic acid is about 2 mg/kg to about 8 mg/kg. In aspects, the dosage of erucic acid is about 2 mg/kg to about 7 mg/kg. In aspects, the dosage of erucic acid is about 2 mg/kg to about 6 mg/kg. In aspects, the dosage of erucic acid is about 2 mg/kg to about 5 mg/kg. In aspects, the dosage of erucic acid is about 2 mg/kg to about 4 mg/kg. In aspects, the dosage of erucic acid is about 2 mg/kg to about 3 mg/kg.

In aspects, the dosage of erucic acid is 2 mg/kg to 25 mg/kg. In aspects, the dosage of erucic acid is 3 mg/kg to 25 mg/kg. In aspects, the dosage of erucic acid is 4 mg/kg to 25 mg/kg. In aspects, the dosage of erucic acid is 5 mg/kg to 25 mg/kg. In aspects, the dosage of erucic acid is 6 mg/kg to 25 mg/kg. In aspects, the dosage of erucic acid is 7 mg/kg to 25 mg/kg. In aspects, the dosage of erucic acid is 8 mg/kg to 25 mg/kg. In aspects, the dosage of erucic acid is 9 mg/kg to 25 mg/kg. In aspects, the dosage of erucic acid is 10 mg/kg to 25 mg/kg. In aspects, the dosage of erucic acid is 11 mg/kg to 25 mg/kg. In aspects, the dosage of erucic acid is 12 mg/kg to 25 mg/kg. In aspects, the dosage of erucic acid is 13 mg/kg to 25 mg/kg. In aspects, the dosage of erucic acid is 14 mg/kg to 25 mg/kg. In aspects, the dosage of erucic acid is 15 mg/kg to 25 mg/kg. In aspects, the dosage of erucic acid is 16 mg/kg to 25 mg/kg. In aspects, the dosage of erucic acid is 17 mg/kg to 25 mg/kg. In aspects, the dosage of erucic acid is 18 mg/kg to 25 mg/kg. In aspects, the dosage of erucic acid is 19 mg/kg to 25 mg/kg. In aspects, the dosage of erucic acid is 20 mg/kg to 25 mg/kg. In aspects, the dosage of erucic acid is 21 mg/kg to 25 mg/kg. In aspects, the dosage of erucic acid is 22 mg/kg to 25 mg/kg. In aspects, the dosage of erucic acid is 23 mg/kg to 25 mg/kg. In aspects, the dosage of erucic acid is 24 mg/kg to 25 mg/kg.

In aspects, the dosage of erucic acid is 2 mg/kg to 24 mg/kg. In aspects, the dosage of erucic acid is 2 mg/kg to 23 mg/kg. In aspects, the dosage of erucic acid is 2 mg/kg to 22 mg/kg. In aspects, the dosage of erucic acid is 2 mg/kg to 21 mg/kg. In aspects, the dosage of erucic acid is 2 mg/kg to 20 mg/kg. In aspects, the dosage of erucic acid is 2 mg/kg to 19 mg/kg. In aspects, the dosage of erucic acid is 2 mg/kg to 18 mg/kg. In aspects, the dosage of erucic acid is 2 mg/kg to 17 mg/kg. In aspects, the dosage of erucic acid is 2 mg/kg to 16 mg/kg. In aspects, the dosage of erucic acid is 2 mg/kg to 15 mg/kg. In aspects, the dosage of erucic acid is 2 mg/kg to 14 mg/kg. In aspects, the dosage of erucic acid is 2 mg/kg to 13 mg/kg. In aspects, the dosage of erucic acid is 2 mg/kg to 12 mg/kg. In aspects, the dosage of erucic acid is 2 mg/kg to 11 mg/kg. In aspects, the dosage of erucic acid is 2 mg/kg to 10 mg/kg. In aspects, the dosage of erucic acid is 2 mg/kg to 9 mg/kg. In aspects, the dosage of erucic acid is 2 mg/kg to 8 mg/kg. In aspects, the dosage of erucic acid is 2 mg/kg to 7 mg/kg. In aspects, the dosage of erucic acid is 2 mg/kg to 6 mg/kg. In aspects, the dosage of erucic acid is 2 mg/kg to 5 mg/kg. In aspects, the dosage of erucic acid is 2 mg/kg to 4 mg/kg. In aspects, the dosage of erucic acid is 2 mg/kg to 3 mg/kg.

In an aspect the dosage of alanine is about 20 mg/kg to about 189 mg/kg. In aspects, the dosage of alanine is about 25 mg/kg to about 189 mg/kg. In aspects, the dosage of alanine is about 30 mg/kg to about 189 mg/kg. In aspects, the dosage of alanine is about 40 mg/kg to about 189 mg/kg. In aspects, the dosage of alanine is about 50 mg/kg to about 189 mg/kg. In aspects, the dosage of alanine is about 75 mg/kg to about 189 mg/kg. In aspects, the dosage of alanine is about 100 mg/kg to about 189 mg/kg. In aspects, the dosage of alanine is about 125 mg/kg to about 189 mg/kg. In aspects, the dosage of alanine is about 150 mg/kg to about 189 mg/kg. In aspects, the dosage of alanine is about 175 mg/kg to about 189 mg/kg. In aspects, the dosage of alanine is about 180 mg/kg to about 189 mg/kg. In aspects, the dosage of alanine is about 185 mg/kg to about 189 mg/kg.

In aspects, the dosage of alanine is about 20 mg/kg to about 185 mg/kg. In aspects, the dosage of alanine is about 20 mg/kg to about 180 mg/kg. In aspects, the dosage of alanine is about 20 mg/kg to about 175 mg/kg. In aspects, the dosage of alanine is about 20 mg/kg to about 150 mg/kg. In aspects, the dosage of alanine is about 20 mg/kg to about 125 mg/kg. In aspects, the dosage of alanine is about 20 mg/kg to about 100 mg/kg. In aspects, the dosage of alanine is about 20 mg/kg to about 75 mg/kg. In aspects, the dosage of alanine is about 20 mg/kg to about 50 mg/kg. In aspects, the dosage of alanine is about 20 mg/kg to about 40 mg/kg. In aspects, the dosage of alanine is about 20 mg/kg to about 30 mg/kg. In aspects, the dosage of alanine is about 20 mg/kg to about 25 mg/kg.

In aspects, the dosage of alanine is 20 mg/kg to 189 mg/kg. In aspects, the dosage of alanine is 25 mg/kg to 189 mg/kg. In aspects, the dosage of alanine is 30 mg/kg to 189 mg/kg. In aspects, the dosage of alanine is 40 mg/kg to 189 mg/kg. In aspects, the dosage of alanine is 50 mg/kg to 189 mg/kg. In aspects, the dosage of alanine is 75 mg/kg to 189 mg/kg. In aspects, the dosage of alanine is 100 mg/kg to 189 mg/kg. In aspects, the dosage of alanine is 125 mg/kg to 189 mg/kg. In aspects, the dosage of alanine is 150 mg/kg to 189 mg/kg. In aspects, the dosage of alanine is 175 mg/kg to 189 mg/kg. In aspects, the dosage of alanine is 180 mg/kg to 189 mg/kg. In aspects, the dosage of alanine is 185 mg/kg to 189 mg/kg.

In aspects, the dosage of alanine is 20 mg/kg to 185 mg/kg. In aspects, the dosage of alanine is 20 mg/kg to 180 mg/kg. In aspects, the dosage of alanine is 20 mg/kg to 175 mg/kg. In aspects, the dosage of alanine is 20 mg/kg to 150 mg/kg. In aspects, the dosage of alanine is 20 mg/kg to 125 mg/kg. In aspects, the dosage of alanine is 20 mg/kg to 100 mg/kg. In aspects, the dosage of alanine is 20 mg/kg to 75 mg/kg. In aspects, the dosage of alanine is 20 mg/kg to 50 mg/kg. In aspects, the dosage of alanine is 20 mg/kg to 40 mg/kg. In aspects, the dosage of alanine is 20 mg/kg to 30 mg/kg. In aspects, the dosage of alanine is 20 mg/kg to 25 mg/kg.

In an aspect the dosage of threonine is about 12 mg/kg to about 130 mg/kg. In aspects, the dosage of threonine is about 13 mg/kg to about 130 mg/kg. In aspects, the dosage of threonine is about 14 mg/kg to about 130 mg/kg. In aspects, the dosage of threonine is about 15 mg/kg to about 130 mg/kg. In aspects, the dosage of threonine is about 20 mg/kg to about 130 mg/kg.

In aspects, the dosage of threonine is about 30 mg/kg to about 130 mg/kg. In aspects, the dosage of threonine is about 40 mg/kg to about 130 mg/kg. In aspects, the dosage of threonine is about 50 mg/kg to about 130 mg/kg. In aspects, the dosage of threonine is about 60 mg/kg to about 130 mg/kg. In aspects, the dosage of threonine is about 70 mg/kg to about 130 mg/kg. In aspects, the dosage of threonine is about 80 mg/kg to about 130 mg/kg. In aspects, the dosage of threonine is about 90 mg/kg to about 130 mg/kg. In aspects, the dosage of threonine is about 100 mg/kg to about 130 mg/kg. In aspects, the dosage of threonine is about 110 mg/kg to about 130 mg/kg. In aspects, the dosage of threonine is about 120 mg/kg to about 130 mg/kg.

In aspects, the dosage of threonine is about 12 mg/kg to about 120 mg/kg. In aspects, the dosage of threonine is about 12 mg/kg to about 110 mg/kg. In aspects, the dosage of threonine is about 12 mg/kg to about 100 mg/kg. In aspects, the dosage of threonine is about 12 mg/kg to about 90 mg/kg. In aspects, the dosage of threonine is about 12 mg/kg to about 80 mg/kg. In aspects, the dosage of threonine is about 12 mg/kg to about 70 mg/kg. In aspects, the dosage of threonine is about 12 mg/kg to about 60 mg/kg. In aspects, the dosage of threonine is about 12 mg/kg to about 50 mg/kg. In aspects, the dosage of threonine is about 12 mg/kg to about 40 mg/kg. In aspects, the dosage of threonine is about 12 mg/kg to about 30 mg/kg. In aspects, the dosage of threonine is about 12 mg/kg to about 20 mg/kg. In aspects, the dosage of threonine is about 12 mg/kg to about 15 mg/kg. In aspects, the dosage of threonine is about 12 mg/kg to about 14 mg/kg. In aspects, the dosage of threonine is about 12 mg/kg to about 13 mg/kg.

In aspects, the dosage of threonine is 12 mg/kg to 130 mg/kg. In aspects, the dosage of threonine is 13 mg/kg to 130 mg/kg. In aspects, the dosage of threonine is 14 mg/kg to 130 mg/kg. In aspects, the dosage of threonine is 15 mg/kg to 130 mg/kg. In aspects, the dosage of threonine is 20 mg/kg to 130 mg/kg. In aspects, the dosage of threonine is 30 mg/kg to 130 mg/kg. In aspects, the dosage of threonine is 40 mg/kg to 130 mg/kg. In aspects, the dosage of threonine is 50 mg/kg to 130 mg/kg. In aspects, the dosage of threonine is 60 mg/kg to 130 mg/kg. In aspects, the dosage of threonine is 70 mg/kg to 130 mg/kg. In aspects, the dosage of threonine is 80 mg/kg to 130 mg/kg. In aspects, the dosage of threonine is 90 mg/kg to 130 mg/kg. In aspects, the dosage of threonine is 100 mg/kg to 130 mg/kg. In aspects, the dosage of threonine is 110 mg/kg to 130 mg/kg. In aspects, the dosage of threonine is 120 mg/kg to 130 mg/kg.

In aspects, the dosage of threonine is 12 mg/kg to 120 mg/kg. In aspects, the dosage of threonine is 12 mg/kg to 110 mg/kg. In aspects, the dosage of threonine is 12 mg/kg to 100 mg/kg. In aspects, the dosage of threonine is 12 mg/kg to 90 mg/kg. In aspects, the dosage of threonine is 12 mg/kg to 80 mg/kg. In aspects, the dosage of threonine is 12 mg/kg to 70 mg/kg. In aspects, the dosage of threonine is 12 mg/kg to 60 mg/kg. In aspects, the dosage of threonine is 12 mg/kg to 50 mg/kg. In aspects, the dosage of threonine is 12 mg/kg to 40 mg/kg. In aspects, the dosage of threonine is 12 mg/kg to 30 mg/kg. In aspects, the dosage of threonine is 12 mg/kg to 20 mg/kg. In aspects, the dosage of threonine is 12 mg/kg to 15 mg/kg. In aspects, the dosage of threonine is 12 mg/kg to 14 mg/kg. In aspects, the dosage of threonine is 12 mg/kg to 13 mg/kg.

In an aspect the dosage of proline is about 24 mg/kg to about 240 mg/kg. In aspects the dosage of proline is about 25 mg/kg to about 240 mg/kg. In aspects the dosage of proline is about 50 mg/kg to about 240 mg/kg. In aspects the dosage of proline is about 75 mg/kg to about 240 mg/kg. In aspects the dosage of proline is about 100 mg/kg to about 240 mg/kg. In aspects the dosage of proline is about 125 mg/kg to about 240 mg/kg. In aspects the dosage of proline is about 150 mg/kg to about 240 mg/kg. In aspects the dosage of proline is about 175 mg/kg to about 240 mg/kg. In aspects the dosage of proline is about 200 mg/kg to about 240 mg/kg. In aspects the dosage of proline is about 225 mg/kg to about 240 mg/kg. In aspects the dosage of proline is about 230 mg/kg to about 240 mg/kg. In aspects the dosage of proline is about 235 mg/kg to about 240 mg/kg.

In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg. In aspects the dosage of proline is about 24 mg/kg to about 235 mg/kg.

In aspects, the dosage of proline is 24 mg/kg to 240 mg/kg. In aspects the dosage of proline is 25 mg/kg to 240 mg/kg. In aspects the dosage of proline is 50 mg/kg to 240 mg/kg. In aspects the dosage of proline is 75 mg/kg to 240 mg/kg. In aspects the dosage of proline is 100 mg/kg to 240 mg/kg. In aspects the dosage of proline is 125 mg/kg to 240 mg/kg. In aspects the dosage of proline is 150 mg/kg to 240 mg/kg. In aspects the dosage of proline is 175 mg/kg to 240 mg/kg. In aspects the dosage of proline is 200 mg/kg to 240 mg/kg. In aspects the dosage of proline is 225 mg/kg to 240 mg/kg. In aspects the dosage of proline is 230 mg/kg to 240 mg/kg. In aspects the dosage of proline is 235 mg/kg to 240 mg/kg.

In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg. In aspects the dosage of proline is 24 mg/kg to 235 mg/kg.

In an aspect the dosage of serine is about 10 mg/kg to about 100 mg/kg. In aspects, the dosage of serine is about 20 mg/kg to about 100 mg/kg. In aspects, the dosage of serine is about 30 mg/kg to about 100 mg/kg. In aspects, the dosage of serine is about 40 mg/kg to about 100 mg/kg. In aspects, the dosage of serine is about 50 mg/kg to about 100 mg/kg. In aspects, the dosage of serine is about 60 mg/kg to about 100 mg/kg. In aspects, the dosage of serine is about 70 mg/kg to about 100 mg/kg. In aspects, the dosage of serine is about 80 mg/kg to about 100 mg/kg. In aspects, the dosage of serine is about 90 mg/kg to about 100 mg/kg.

In aspects, the dosage of serine is about 10 mg/kg to about 90 mg/kg. In aspects, the dosage of serine is about 10 mg/kg to about 80 mg/kg. In aspects, the dosage of serine is about 10 mg/kg to about 70 mg/kg. In aspects, the dosage of serine is about 10 mg/kg to about 60 mg/kg. In aspects, the dosage of serine is about 10 mg/kg to about 50 mg/kg. In aspects, the dosage of serine is about 10 mg/kg to about 40 mg/kg. In aspects, the dosage of serine is about 10 mg/kg to about 30 mg/kg. In aspects, the dosage of serine is about 10 mg/kg to about 20 mg/kg.

In aspects, the dosage of serine is 10 mg/kg to 100 mg/kg. In aspects, the dosage of serine is 20 mg/kg to 100 mg/kg. In aspects, the dosage of serine is 30 mg/kg to 100 mg/kg. In aspects, the dosage of serine is 40 mg/kg to 100 mg/kg. In aspects, the dosage of serine is 50 mg/kg to 100 mg/kg. In aspects, the dosage of serine is 60 mg/kg to 100 mg/kg. In aspects, the dosage of serine is 70 mg/kg to 100 mg/kg. In aspects, the dosage of serine is 80 mg/kg to 100 mg/kg. In aspects, the dosage of serine is 90 mg/kg to 100 mg/kg.

In aspects, the dosage of serine is 10 mg/kg to 90 mg/kg. In aspects, the dosage of serine is 10 mg/kg to 80 mg/kg. In aspects, the dosage of serine is 10 mg/kg to 70 mg/kg. In aspects, the dosage of serine is 10 mg/kg to 60 mg/kg. In aspects, the dosage of serine is 10 mg/kg to 50 mg/kg. In aspects, the dosage of serine is 10 mg/kg to 40 mg/kg. In aspects, the dosage of serine is 10 mg/kg to 30 mg/kg. In aspects, the dosage of serine is 10 mg/kg to 20 mg/kg.

In an aspect the dosage of leucine is about 28 mg/kg to about 310 mg/kg. In aspects, the dosage of leucine is about 29 mg/kg to about 310 mg/kg. In aspects, the dosage of leucine is about 30 mg/kg to about 310 mg/kg. In aspects, the dosage of leucine is about 40 mg/kg to about 310 mg/kg. In aspects, the dosage of leucine is about 50 mg/kg to about 310 mg/kg. In aspects, the dosage of leucine is about 100 mg/kg to about 310 mg/kg. In aspects, the dosage of leucine is about 150 mg/kg to about 310 mg/kg. In aspects, the dosage of leucine is about 200 mg/kg to about 310 mg/kg. In aspects, the dosage of leucine is about 250 mg/kg to about 310 mg/kg. In aspects, the dosage of leucine is about 300 mg/kg to about 310 mg/kg. In aspects, the dosage of leucine is about 305 mg/kg to about 310 mg/kg.

In aspects, the dosage of leucine is about 28 mg/kg to about 305 mg/kg. In aspects, the dosage of leucine is about 28 mg/kg to about 300 mg/kg. In aspects, the dosage of leucine is about 28 mg/kg to about 250 mg/kg. In aspects, the dosage of leucine is about 28 mg/kg to about 200 mg/kg. In aspects, the dosage of leucine is about 28 mg/kg to about 150 mg/kg. In aspects, the dosage of leucine is about 28 mg/kg to about 100 mg/kg. In aspects, the dosage of leucine is about 28 mg/kg to about 50 mg/kg. In aspects, the dosage of leucine is about 28 mg/kg to about 40 mg/kg. In aspects, the dosage of leucine is about 28 mg/kg to about 30 mg/kg. In aspects, the dosage of leucine is about 28 mg/kg to about 29 mg/kg.

In aspects, the dosage of leucine is 28 mg/kg to 310 mg/kg. In aspects, the dosage of leucine is 29 mg/kg to 310 mg/kg. In aspects, the dosage of leucine is 30 mg/kg to 310 mg/kg. In aspects, the dosage of leucine is 40 mg/kg to 310 mg/kg. In aspects, the dosage of leucine is 50 mg/kg to 310 mg/kg. In aspects, the dosage of leucine is 100 mg/kg to 310 mg/kg. In aspects, the dosage of leucine is 150 mg/kg to 310 mg/kg. In aspects, the dosage of leucine is 200 mg/kg to 310 mg/kg. In aspects, the dosage of leucine is 250 mg/kg to 310 mg/kg. In aspects, the dosage of leucine is 300 mg/kg to 310 mg/kg. In aspects, the dosage of leucine is 305 mg/kg to 310 mg/kg.

In aspects, the dosage of leucine is 28 mg/kg to 305 mg/kg. In aspects, the dosage of leucine is 28 mg/kg to 300 mg/kg. In aspects, the dosage of leucine is 28 mg/kg to 250 mg/kg. In aspects, the dosage of leucine is 28 mg/kg to 200 mg/kg. In aspects, the dosage of leucine is 28 mg/kg to 150 mg/kg. In aspects, the dosage of leucine is 28 mg/kg to 100 mg/kg. In aspects, the dosage of leucine is 28 mg/kg to 50 mg/kg. In aspects, the dosage of leucine is 28 mg/kg to 40 mg/kg. In aspects, the dosage of leucine is 28 mg/kg to 30 mg/kg. In aspects, the dosage of leucine is 28 mg/kg to 29 mg/kg.

In an aspect the dosage of isoleucine is about 24 mg/kg to about 245 mg/kg. In aspects, the dosage of isoleucine is about 25 mg/kg to about 245 mg/kg. In aspects, the dosage of isoleucine is about 50 mg/kg to about 245 mg/kg. In aspects, the dosage of isoleucine is about 75 mg/kg to about 245 mg/kg. In aspects, the dosage of isoleucine is about 100 mg/kg to about 245 mg/kg. In aspects, the dosage of isoleucine is about 125 mg/kg to about 245 mg/kg. In aspects, the dosage of isoleucine is about 150 mg/kg to about 245 mg/kg. In aspects, the dosage of isoleucine is about 175 mg/kg to about 245 mg/kg. In aspects, the dosage of isoleucine is about 200 mg/kg to about 245 mg/kg. In aspects, the dosage of isoleucine is about 225 mg/kg to about 245 mg/kg. In aspects, the dosage of isoleucine is about 240 mg/kg to about 245 mg/kg.

In aspects, the dosage of isoleucine is about 24 mg/kg to about 240 mg/kg. In aspects, the dosage of isoleucine is about 24 mg/kg to about 225 mg/kg. In aspects, the dosage of isoleucine is about 24 mg/kg to about 200 mg/kg. In aspects, the dosage of isoleucine is about 24 mg/kg to about 175 mg/kg. In aspects, the dosage of isoleucine is about 24 mg/kg to about 150 mg/kg. In aspects, the dosage of isoleucine is about 24 mg/kg to about 125 mg/kg. In aspects, the dosage of isoleucine is about 24 mg/kg to about 100 mg/kg. In aspects, the dosage of isoleucine is about 24 mg/kg to about 75 mg/kg. In aspects, the dosage of isoleucine is about 24 mg/kg to about 50 mg/kg. In aspects, the dosage of isoleucine is about 24 mg/kg to about 25 mg/kg.

In aspects, the dosage of isoleucine is 24 mg/kg to 245 mg/kg. In aspects, the dosage of isoleucine is 25 mg/kg to 245 mg/kg. In aspects, the dosage of isoleucine is 50 mg/kg to 245 mg/kg. In aspects, the dosage of isoleucine is 75 mg/kg to 245 mg/kg. In aspects, the dosage of isoleucine is 100 mg/kg to 245 mg/kg. In aspects, the dosage of isoleucine is 125 mg/kg to 245 mg/kg. In aspects, the dosage of isoleucine is 150 mg/kg to 245 mg/kg. In aspects, the dosage of isoleucine is 175 mg/kg to 245 mg/kg. In aspects, the dosage of isoleucine is 200 mg/kg to 245 mg/kg. In aspects, the dosage of isoleucine is 225 mg/kg to about 245 mg/kg. In aspects, the dosage of isoleucine is 240 mg/kg to 245 mg/kg.

In aspects, the dosage of isoleucine is 24 mg/kg to 240 mg/kg. In aspects, the dosage of isoleucine is 24 mg/kg to 225 mg/kg. In aspects, the dosage of isoleucine is 24 mg/kg to 200 mg/kg. In aspects, the dosage of isoleucine is 24 mg/kg to 175 mg/kg. In aspects, the dosage of isoleucine is 24 mg/kg to 150 mg/kg. In aspects, the dosage of isoleucine is 24 mg/kg to 125 mg/kg. In aspects, the dosage of isoleucine is 24 mg/kg to 100 mg/kg. In aspects, the dosage of isoleucine is 24 mg/kg to 75 mg/kg. In aspects, the dosage of isoleucine is 24 mg/kg to 50 mg/kg. In aspects, the dosage of isoleucine is 24 mg/kg to 25 mg/kg.

In an aspect the dosage of valine is about 22 mg/kg to about 230 mg/kg. In aspects, the dosage of valine is about 23 mg/kg to about 230 mg/kg. In aspects, the dosage of valine is about 24 mg/kg to about 230 mg/kg. In aspects, the dosage of valine is about 25 mg/kg to about 230 mg/kg. In aspects, the dosage of valine is about 50 mg/kg to about 230 mg/kg. In aspects, the dosage of valine is about 75 mg/kg to about 230 mg/kg. In aspects, the dosage of valine is about 100 mg/kg to about 230 mg/kg. In aspects, the dosage of valine is about 125 mg/kg to about 230 mg/kg. In aspects, the dosage of valine is about 150 mg/kg to about 230 mg/kg. In aspects, the dosage of valine is about 175 mg/kg to about 230 mg/kg. In aspects, the dosage of valine is about 200 mg/kg to about 230 mg/kg. In aspects, the dosage of valine is about 225 mg/kg to about 230 mg/kg.

In aspects, the dosage of valine is about 22 mg/kg to about 225 mg/kg. In aspects, the dosage of valine is about 22 mg/kg to about 200 mg/kg. In aspects, the dosage of valine is about 22 mg/kg to about 175 mg/kg. In aspects, the dosage of valine is about 22 mg/kg to about 150 mg/kg. In aspects, the dosage of valine is about 22 mg/kg to about 125 mg/kg. In aspects, the dosage of valine is about 22 mg/kg to about 100 mg/kg. In aspects, the dosage of valine is about 22 mg/kg to about 75 mg/kg. In aspects, the dosage of valine is about 22 mg/kg to about 50 mg/kg. In aspects, the dosage of valine is about 22 mg/kg to about 25 mg/kg. In aspects, the dosage of valine is about 22 mg/kg to about 24 mg/kg. In aspects, the dosage of valine is about 22 mg/kg to about 23 mg/kg.

In aspects, the dosage of valine is 22 mg/kg to 230 mg/kg. In aspects, the dosage of valine is 23 mg/kg to 230 mg/kg. In aspects, the dosage of valine is 24 mg/kg to 230 mg/kg. In aspects, the dosage of valine is 25 mg/kg to 230 mg/kg. In aspects, the dosage of valine is 50 mg/kg to 230 mg/kg. In aspects, the dosage of valine is 75 mg/kg to 230 mg/kg. In aspects, the dosage of valine is 100 mg/kg to 230 mg/kg. In aspects, the dosage of valine is 125 mg/kg to 230 mg/kg. In aspects, the dosage of valine is 150 mg/kg to 230 mg/kg. In aspects, the dosage of valine is 175 mg/kg to 230 mg/kg. In aspects, the dosage of valine is 200 mg/kg to 230 mg/kg. In aspects, the dosage of valine is 225 mg/kg to 230 mg/kg.

In aspects, the dosage of valine is 22 mg/kg to 225 mg/kg. In aspects, the dosage of valine is 22 mg/kg to 200 mg/kg. In aspects, the dosage of valine is 22 mg/kg to 175 mg/kg. In aspects, the dosage of valine is 22 mg/kg to 150 mg/kg. In aspects, the dosage of valine is 22 mg/kg to 125 mg/kg. In aspects, the dosage of valine is 22 mg/kg to 100 mg/kg. In aspects, the dosage of valine is 22 mg/kg to 75 mg/kg. In aspects, the dosage of valine is 22 mg/kg to 50 mg/kg. In aspects, the dosage of valine is 22 mg/kg to 25 mg/kg. In aspects, the dosage of valine is 22 mg/kg to 24 mg/kg. In aspects, the dosage of valine is 22 mg/kg to 23 mg/kg.

In an aspect the dosage of phenylalanine is about 15 mg/kg to about 150 mg/kg. In aspects, the dosage of phenylalanine is about 20 mg/kg to about 150 mg/kg. In aspects, the dosage of phenylalanine is about 30 mg/kg to about 150 mg/kg. In aspects, the dosage of phenylalanine is about 40 mg/kg to about 150 mg/kg. In aspects, the dosage of phenylalanine is about 50 mg/kg to about 150 mg/kg. In aspects, the dosage of phenylalanine is about 60 mg/kg to about 150 mg/kg. In aspects, the dosage of phenylalanine is about 70 mg/kg to about 150 mg/kg. In aspects, the dosage of phenylalanine is about 80 mg/kg to about 150 mg/kg. In aspects, the dosage of phenylalanine is about 90 mg/kg to about 150 mg/kg. In aspects, the dosage of phenylalanine is about 100 mg/kg to about 150 mg/kg. In aspects, the dosage of phenylalanine is about 110 mg/kg to about 150 mg/kg. In aspects, the dosage of phenylalanine is about 120 mg/kg to about 150 mg/kg. In aspects, the dosage of phenylalanine is about 130 mg/kg to about 150 mg/kg. In aspects, the dosage of phenylalanine is about 140 mg/kg to about 150 mg/kg.

In aspects, the dosage of phenylalanine is about 15 mg/kg to about 140 mg/kg. In aspects, the dosage of phenylalanine is about 15 mg/kg to about 130 mg/kg. In aspects, the dosage of phenylalanine is about 15 mg/kg to about 120 mg/kg. In aspects, the dosage of phenylalanine is about 15 mg/kg to about 110 mg/kg. In aspects, the dosage of phenylalanine is about 15 mg/kg to about 100 mg/kg. In aspects, the dosage of phenylalanine is about 15 mg/kg to about 90 mg/kg. In aspects, the dosage of phenylalanine is about 15 mg/kg to about 80 mg/kg. In aspects, the dosage of phenylalanine is about 15 mg/kg to about 70 mg/kg. In aspects, the dosage of phenylalanine is about 15 mg/kg to about 60 mg/kg. In aspects, the dosage of phenylalanine is about 15 mg/kg to about 50 mg/kg. In aspects, the dosage of phenylalanine is about 15 mg/kg to about 40 mg/kg. In aspects, the dosage of phenylalanine is about 15 mg/kg to about 30 mg/kg. In aspects, the dosage of phenylalanine is about 15 mg/kg to about 20 mg/kg.

In an aspect the dosage of phenylalanine is 15 mg/kg to 150 mg/kg. In aspects, the dosage of phenylalanine is 20 mg/kg to 150 mg/kg. In aspects, the dosage of phenylalanine is 30 mg/kg to 150 mg/kg. In aspects, the dosage of phenylalanine is 40 mg/kg to 150 mg/kg. In aspects, the dosage of phenylalanine is 50 mg/kg to 150 mg/kg. In aspects, the dosage of phenylalanine is 60 mg/kg to 150 mg/kg. In aspects, the dosage of phenylalanine is 70 mg/kg to 150 mg/kg.

In aspects, the dosage of phenylalanine is 80 mg/kg to 150 mg/kg. In aspects, the dosage of phenylalanine is 90 mg/kg to 150 mg/kg. In aspects, the dosage of phenylalanine is 100 mg/kg to 150 mg/kg. In aspects, the dosage of phenylalanine is 110 mg/kg to 150 mg/kg. In aspects, the dosage of phenylalanine is 120 mg/kg to 150 mg/kg. In aspects, the dosage of phenylalanine is 130 mg/kg to 150 mg/kg. In aspects, the dosage of phenylalanine is 140 mg/kg to 150 mg/kg.

In aspects, the dosage of phenylalanine is 15 mg/kg to 140 mg/kg. In aspects, the dosage of phenylalanine is 15 mg/kg to 130 mg/kg. In aspects, the dosage of phenylalanine is 15 mg/kg to 120 mg/kg. In aspects, the dosage of phenylalanine is 15 mg/kg to 110 mg/kg. In aspects, the dosage of phenylalanine is 15 mg/kg to 100 mg/kg. In aspects, the dosage of phenylalanine is 15 mg/kg to 90 mg/kg. In aspects, the dosage of phenylalanine is 15 mg/kg to 80 mg/kg. In aspects, the dosage of phenylalanine is 15 mg/kg to 70 mg/kg. In aspects, the dosage of phenylalanine is 15 mg/kg to 60 mg/kg. In aspects, the dosage of phenylalanine is 15 mg/kg to 50 mg/kg. In aspects, the dosage of phenylalanine is 15 mg/kg to 40 mg/kg. In aspects, the dosage of phenylalanine is 15 mg/kg to 30 mg/kg. In aspects, the dosage of phenylalanine is 15 mg/kg to 20 mg/kg.

In an aspect the dosage of tyrosine is about 9 mg/kg to about 90 mg/kg. In aspects, the dosage of tyrosine is about 10 mg/kg to about 90 mg/kg. In aspects, the dosage of tyrosine is about 20 mg/kg to about 90 mg/kg. In aspects, the dosage of tyrosine is about 30 mg/kg to about 90 mg/kg. In aspects, the dosage of tyrosine is about 40 mg/kg to about 90 mg/kg. In aspects, the dosage of tyrosine is about 50 mg/kg to about 90 mg/kg. In aspects, the dosage of tyrosine is about 60 mg/kg to about 90 mg/kg. In aspects, the dosage of tyrosine is about 70 mg/kg to about 90 mg/kg. In aspects, the dosage of tyrosine is about 80 mg/kg to about 90 mg/kg.

In aspects, the dosage of tyrosine is about 9 mg/kg to about 80 mg/kg. In aspects, the dosage of tyrosine is about 9 mg/kg to about 70 mg/kg. In aspects, the dosage of tyrosine is about 9 mg/kg to about 60 mg/kg. In aspects, the dosage of tyrosine is about 9 mg/kg to about 50 mg/kg. In aspects, the dosage of tyrosine is about 9 mg/kg to about 40 mg/kg. In aspects, the dosage of tyrosine is about 9 mg/kg to about 30 mg/kg. In aspects, the dosage of tyrosine is about 9 mg/kg to about 20 mg/kg. In aspects, the dosage of tyrosine is about 9 mg/kg to about 10 mg/kg.

In aspects, the dosage of tyrosine is 9 mg/kg to 90 mg/kg. In aspects, the dosage of tyrosine is 10 mg/kg to 90 mg/kg. In aspects, the dosage of tyrosine is 20 mg/kg to 90 mg/kg. In aspects, the dosage of tyrosine is 30 mg/kg to 90 mg/kg. In aspects, the dosage of tyrosine is 40 mg/kg to 90 mg/kg. In aspects, the dosage of tyrosine is 50 mg/kg to 90 mg/kg. In aspects, the dosage of tyrosine is 60 mg/kg to 90 mg/kg. In aspects, the dosage of tyrosine is 70 mg/kg to 90 mg/kg. In aspects, the dosage of tyrosine is 80 mg/kg to 90 mg/kg.

In aspects, the dosage of tyrosine is 9 mg/kg to 80 mg/kg. In aspects, the dosage of tyrosine is 9 mg/kg to 70 mg/kg. In aspects, the dosage of tyrosine is 9 mg/kg to 60 mg/kg. In aspects, the dosage of tyrosine is 9 mg/kg to 50 mg/kg. In aspects, the dosage of tyrosine is 9 mg/kg to 40 mg/kg. In aspects, the dosage of tyrosine is 9 mg/kg to 30 mg/kg. In aspects, the dosage of tyrosine is 9 mg/kg to 20 mg/kg. In aspects, the dosage of tyrosine is 9 mg/kg to 10 mg/kg.

In an aspect the dosage of methionine is about 6 mg/kg to about 60 mg/kg. In aspects, the dosage of methionine is about 7 mg/kg to about 60 mg/kg. In aspects, the dosage of methionine is about 8 mg/kg to about 60 mg/kg. In aspects, the dosage of methionine is about 9 mg/kg to about 60 mg/kg. In aspects, the dosage of methionine is about 10 mg/kg to about 60 mg/kg. In aspects, the dosage of methionine is about 20 mg/kg to about 60 mg/kg. In aspects, the dosage of methionine is about 30 mg/kg to about 60 mg/kg. In aspects, the dosage of methionine is about 40 mg/kg to about 60 mg/kg. In aspects, the dosage of methionine is about 50 mg/kg to about 60 mg/kg.

In aspects, the dosage of methionine is about 6 mg/kg to about 50 mg/kg. In aspects, the dosage of methionine is about 6 mg/kg to about 40 mg/kg. In aspects, the dosage of methionine is about 6 mg/kg to about 30 mg/kg. In aspects, the dosage of methionine is about 6 mg/kg to about 20 mg/kg. In aspects, the dosage of methionine is about 6 mg/kg to about 10 mg/kg. In aspects, the dosage of methionine is about 6 mg/kg to about 9 mg/kg. In aspects, the dosage of methionine is about 6 mg/kg to about 8 mg/kg. In aspects, the dosage of methionine is about 6 mg/kg to about 7 mg/kg.

In an aspect the dosage of methionine is 6 mg/kg to 60 mg/kg. In aspects, the dosage of methionine is 7 mg/kg to 60 mg/kg. In aspects, the dosage of methionine is 8 mg/kg to 60 mg/kg. In aspects, the dosage of methionine is 9 mg/kg to 60 mg/kg. In aspects, the dosage of methionine is 10 mg/kg to 60 mg/kg. In aspects, the dosage of methionine is 20 mg/kg to 60 mg/kg. In aspects, the dosage of methionine is 30 mg/kg to 60 mg/kg. In aspects, the dosage of methionine is 40 mg/kg to 60 mg/kg. In aspects, the dosage of methionine is 50 mg/kg to 60 mg/kg.

In aspects, the dosage of methionine is 6 mg/kg to 50 mg/kg. In aspects, the dosage of methionine is 6 mg/kg to 40 mg/kg. In aspects, the dosage of methionine is 6 mg/kg to 30 mg/kg. In aspects, the dosage of methionine is 6 mg/kg to 20 mg/kg. In aspects, the dosage of methionine is 6 mg/kg to 10 mg/kg. In aspects, the dosage of methionine is 6 mg/kg to 9 mg/kg. In aspects, the dosage of methionine is 6 mg/kg to 8 mg/kg. In aspects, the dosage of methionine is 6 mg/kg to 7 mg/kg.

In an aspect the dosage of cysteine is about 4 mg/kg to about 45 mg/kg. In aspects, the dosage of cysteine is about 5 mg/kg to about 45 mg/kg. In aspects, the dosage of cysteine is about 10 mg/kg to about 45 mg/kg. In aspects, the dosage of cysteine is about 15 mg/kg to about 45 mg/kg. In aspects, the dosage of cysteine is about 20 mg/kg to about 45 mg/kg. In aspects, the dosage of cysteine is about 25 mg/kg to about 45 mg/kg. In aspects, the dosage of cysteine is about 30 mg/kg to about 45 mg/kg. In aspects, the dosage of cysteine is about 35 mg/kg to about 45 mg/kg. In aspects, the dosage of cysteine is about 40 mg/kg to about 45 mg/kg.

In aspects, the dosage of cysteine is about 4 mg/kg to about 40 mg/kg. In aspects, the dosage of cysteine is about 4 mg/kg to about 35 mg/kg. In aspects, the dosage of cysteine is about 4 mg/kg to about 30 mg/kg. In aspects, the dosage of cysteine is about 4 mg/kg to about 25 mg/kg. In aspects, the dosage of cysteine is about 4 mg/kg to about 20 mg/kg. In aspects, the dosage of cysteine is about 4 mg/kg to about 15 mg/kg. In aspects, the dosage of cysteine is about 4 mg/kg to about 10 mg/kg. In aspects, the dosage of cysteine is about 4 mg/kg to about 5 mg/kg.

In aspects, the dosage of cysteine is 4 mg/kg to 45 mg/kg. In aspects, the dosage of cysteine is 5 mg/kg to 45 mg/kg. In aspects, the dosage of cysteine is 10 mg/kg to 45 mg/kg. In aspects, the dosage of cysteine is 15 mg/kg to 45 mg/kg. In aspects, the dosage of cysteine is 20 mg/kg to 45 mg/kg. In aspects, the dosage of cysteine is 25 mg/kg to 45 mg/kg. In aspects, the dosage of cysteine is 30 mg/kg to 45 mg/kg. In aspects, the dosage of cysteine is 35 mg/kg to 45 mg/kg. In aspects, the dosage of cysteine is 40 mg/kg to 45 mg/kg.

In aspects, the dosage of cysteine is 4 mg/kg to 40 mg/kg. In aspects, the dosage of cysteine is 4 mg/kg to 35 mg/kg. In aspects, the dosage of cysteine is 4 mg/kg to 30 mg/kg. In aspects, the dosage of cysteine is 4 mg/kg to 25 mg/kg. In aspects, the dosage of cysteine is 4 mg/kg to 20 mg/kg. In aspects, the dosage of cysteine is 4 mg/kg to 15 mg/kg. In aspects, the dosage of cysteine is 4 mg/kg to 10 mg/kg. In aspects, the dosage of cysteine is 4 mg/kg to 5 mg/kg.

In an aspect the dosage of tryptophan is about 6 mg/kg to about 61 mg/kg. In aspects, the dosage of tryptophan is about 7 mg/kg to about 61 mg/kg. In aspects, the dosage of tryptophan is about 8 mg/kg to about 61 mg/kg. In aspects, the dosage of tryptophan is about 9 mg/kg to about 61 mg/kg. In aspects, the dosage of tryptophan is about 10 mg/kg to about 61 mg/kg. In aspects, the dosage of tryptophan is about 15 mg/kg to about 61 mg/kg. In aspects, the dosage of tryptophan is about 20 mg/kg to about 61 mg/kg. In aspects, the dosage of tryptophan is about 25 mg/kg to about 61 mg/kg. In aspects, the dosage of tryptophan is about 30 mg/kg to about 61 mg/kg. In aspects, the dosage of tryptophan is about 35 mg/kg to about 61 mg/kg. In aspects, the dosage of tryptophan is about 40 mg/kg to about 61 mg/kg. In aspects, the dosage of tryptophan is about 45 mg/kg to about 61 mg/kg. In aspects, the dosage of tryptophan is about 50 mg/kg to about 61 mg/kg. In aspects, the dosage of tryptophan is about 55 mg/kg to about 61 mg/kg. In aspects, the dosage of tryptophan is about 60 mg/kg to about 61 mg/kg.

In aspects, the dosage of tryptophan is about 6 mg/kg to about 60 mg/kg. In aspects, the dosage of tryptophan is about 6 mg/kg to about 55 mg/kg. In aspects, the dosage of tryptophan is about 6 mg/kg to about 50 mg/kg. In aspects, the dosage of tryptophan is about 6 mg/kg to about 45 mg/kg. In aspects, the dosage of tryptophan is about 6 mg/kg to about 40 mg/kg. In aspects, the dosage of tryptophan is about 6 mg/kg to about 35 mg/kg. In aspects, the dosage of tryptophan is about 6 mg/kg to about 30 mg/kg. In aspects, the dosage of tryptophan is about 6 mg/kg to about 25 mg/kg. In aspects, the dosage of tryptophan is about 6 mg/kg to about 20 mg/kg. In aspects, the dosage of tryptophan is about 6 mg/kg to about 15 mg/kg. In aspects, the dosage of tryptophan is about 6 mg/kg to about 10 mg/kg. In aspects, the dosage of tryptophan is about 6 mg/kg to about 9 mg/kg. In aspects, the dosage of tryptophan is about 6 mg/kg to about 8 mg/kg. In aspects, the dosage of tryptophan is about 6 mg/kg to about 7 mg/kg.

In an aspect the dosage of tryptophan is 6 mg/kg to 61 mg/kg. In aspects, the dosage of tryptophan is 7 mg/kg to 61 mg/kg. In aspects, the dosage of tryptophan is 8 mg/kg to 61 mg/kg. In aspects, the dosage of tryptophan is 9 mg/kg to 61 mg/kg. In aspects, the dosage of tryptophan is 10 mg/kg to 61 mg/kg. In aspects, the dosage of tryptophan is 15 mg/kg to 61 mg/kg. In aspects, the dosage of tryptophan is 20 mg/kg to 61 mg/kg. In aspects, the dosage of tryptophan is 25 mg/kg to 61 mg/kg. In aspects, the dosage of tryptophan is 30 mg/kg to 61 mg/kg. In aspects, the dosage of tryptophan is 35 mg/kg to 61 mg/kg. In aspects, the dosage of tryptophan is 40 mg/kg to 61 mg/kg. In aspects, the dosage of tryptophan is 45 mg/kg to 61 mg/kg. In aspects, the dosage of tryptophan is 50 mg/kg to 61 mg/kg. In aspects, the dosage of tryptophan is 55 mg/kg to 61 mg/kg. In aspects, the dosage of tryptophan is 60 mg/kg to 61 mg/kg.

In aspects, the dosage of tryptophan is 6 mg/kg to 60 mg/kg. In aspects, the dosage of tryptophan is 6 mg/kg to 55 mg/kg. In aspects, the dosage of tryptophan is 6 mg/kg to 50 mg/kg. In aspects, the dosage of tryptophan is 6 mg/kg to 45 mg/kg. In aspects, the dosage of tryptophan is 6 mg/kg to 40 mg/kg. In aspects, the dosage of tryptophan is 6 mg/kg to 35 mg/kg. In aspects, the dosage of tryptophan is 6 mg/kg to 30 mg/kg. In aspects, the dosage of tryptophan is 6 mg/kg to 25 mg/kg. In aspects, the dosage of tryptophan is 6 mg/kg to 20 mg/kg. In aspects, the dosage of tryptophan is 6 mg/kg to 15 mg/kg. In aspects, the dosage of tryptophan is 6 mg/kg to 10 mg/kg. In aspects, the dosage of tryptophan is 6 mg/kg to 9 mg/kg. In aspects, the dosage of tryptophan is 6 mg/kg to 8 mg/kg. In aspects, the dosage of tryptophan is 6 mg/kg to 7 mg/kg.

In an aspect the method further includes administering an effective amount of an agent to the subject, wherein the agent is administered between about 1 day and 7 days after a traumatic pain event and wherein the agent is an NAAA (N-Acylethanolamine acid amidase) inhibitor, FAAH (Fatty acid amide hydrolase) inhibitor, PPARα (peroxisome proliferator-activated receptor-α) agonist, acetyl-L-carnitine, α-lipoic acid, or olesoxime. In embodiments, the agent is administered between about 24 hours and about 96 hours after the traumatic pain event. In embodiments, the agent is an NAAA (N-Acylethanolamine acid amidase) inhibitor. In embodiments, the agent is a FAAH (Fatty acid amide hydrolase) inhibitor. In embodiments, the agent is a PPARα (peroxisome proliferator-activated receptor-α) agonist. In embodiments, the agent is PEA (palmitoylethanolamide). In embodiments, the agent is acetyl-L-carnitine. In embodiments, the agent is α-lipoic acid. In embodiments, the agent is olesoxime. In embodiments, the agent is administered in a therapeutically effective amount.

In embodiments, the pain is neuropathic pain, nociceptive pain, chronic pain, neuropathy glossopharyngeal neuralgia, occipital neuralgia, postherpetic neuralgia, trigeminal neuralgia, post herpetic neuralgia, trigeminal neuralgia, causalgia, diabetic neuropathy, complex regional pain syndrome (CRPS), neurogenic pain, peripheral pain, polyneuropathic pain, toxic neuropathy, chronic neuropathy or pruritus, or acute postoperative pain.

In embodiments, the composition prevents the transition from an acute pain state to a chronic pain state. In embodiments, the composition prevents chronic pain in a subject with acute pain.

In embodiments, the subject is a cancer patient. In embodiments, the subject is a diabetic patient.

In an aspect is provided a method of preventing chronic pain in a subject, the method including administering preoperatively or perioperatively an effective amount of the composition to the subject.

In embodiments, the method includes administering the composition one to four times per day. In embodiments, the method includes administering the composition one time a day. In embodiments, the method includes administering the composition two times a day. In embodiments, the method includes administering the composition three times a day.

In embodiments, the method includes administering the composition between about 0 days and about 30 days after the traumatic pain event. In embodiments, the method includes administering the composition between about 1 days and about 30 days after the traumatic pain event. In embodiments, the method includes administering the composition between about 2 days and about 30 days after the traumatic pain event. In embodiments, the method includes administering the composition between about 3 days and about 30 days after the traumatic pain event. In embodiments, the method includes administering the composition between about 4 days and about 30 days after the traumatic pain event. In embodiments, the method includes administering the composition between about 5 days and about 30 days after the traumatic pain event. In embodiments, the method includes administering the composition between about 6 days and about 30 days after the traumatic pain event. In embodiments, the method includes administering the composition between about 7 days and about 30 days after the traumatic pain event. In embodiments, the method includes administering the composition between about 8 days and about 30 days after the traumatic pain event. In embodiments, the method includes administering the composition between about 9 days and about 30 days after the traumatic pain event. In embodiments, the method includes administering the composition between about 10 days and about 30 days after the traumatic pain event. In embodiments, the method includes administering the composition between about 15 days and about 30 days after the traumatic pain event. In embodiments, the method includes administering the composition between about 20 days and about 30 days after the traumatic pain event. In embodiments, the method includes administering the composition between about 25 days and about 30 days after the traumatic pain event.

In embodiments, the method includes administering the composition between about 0 days and about 25 days after the traumatic pain event. In embodiments, the method includes administering the composition between about 0 days and about 20 days after the traumatic pain event. In embodiments, the method includes administering the composition between about 0 days and about 15 days after the traumatic pain event. In embodiments, the method includes administering the composition between about 0 days and about 10 days after the traumatic pain event. In embodiments, the method includes administering the composition between about 0 days and about 9 days after the traumatic pain event. In embodiments, the method includes administering the composition between about 0 days and about 8 days after the traumatic pain event. In embodiments, the method includes administering the composition between about 0 days and about 7 days after the traumatic pain event. In embodiments, the method includes administering the composition between about 0 days and about 6 days after the traumatic pain event. In embodiments, the method includes administering the composition between about 0 days and about 5 days after the traumatic pain event. In embodiments, the method includes administering the composition between about 0 days and about 4 days after the traumatic pain event. In embodiments, the method includes administering the composition between about 0 days and about 3 days after the traumatic pain event. In embodiments, the method includes administering the composition between about 0 days and about 2 days after the traumatic pain event. In embodiments, the method includes administering the composition between about 0 days and about 1 days after the traumatic pain event.

In embodiments, the method includes administering the composition between 0 days and 30 days after the traumatic pain event. In embodiments, the method includes administering the composition between 1 days and 30 days after the traumatic pain event. In embodiments, the method includes administering the composition between 2 days and 30 days after the traumatic pain event. In embodiments, the method includes administering the composition between 3 days and 30 days after the traumatic pain event. In embodiments, the method includes administering the composition between 4 days and 30 days after the traumatic pain event. In embodiments, the method includes administering the composition between 5 days and 30 days after the traumatic pain event. In embodiments, the method includes administering the composition between 6 days and 30 days after the traumatic pain event. In embodiments, the method includes administering the composition between 7 days and 30 days after the traumatic pain event. In embodiments, the method includes administering the composition between about 8 days and about 30 days after the traumatic pain event. In embodiments, the method includes administering the composition between 9 days and 30 days after the traumatic pain event. In embodiments, the method includes administering the composition between 10 days and 30 days after the traumatic pain event. In embodiments, the method includes administering the composition between 15 days and 30 days after the traumatic pain event. In embodiments, the method includes administering the composition between 20 days and 30 days after the traumatic pain event. In embodiments, the method includes administering the composition between 25 days and 30 days after the traumatic pain event.

In embodiments, the method includes administering the composition between 0 days and 25 days after the traumatic pain event. In embodiments, the method includes administering the composition between 0 days and 20 days after the traumatic pain event. In embodiments, the method includes administering the composition between 0 days and 15 days after the traumatic pain event. In embodiments, the method includes administering the composition between 0 days and 10 days after the traumatic pain event. In embodiments, the method includes administering the composition between 0 days and 9 days after the traumatic pain event. In embodiments, the method includes administering the composition between 0 days and 8 days after the traumatic pain event. In embodiments, the method includes administering the composition between 0 days and 7 days after the traumatic pain event. In embodiments, the method includes administering the composition between 0 days and 6 days after the traumatic pain event. In embodiments, the method includes administering the composition between 0 days and 5 days after the traumatic pain event. In embodiments, the method includes administering the composition between 0 days and 4 days after the traumatic pain event. In embodiments, the method includes administering the composition between 0 days and 3 days after the traumatic pain event. In embodiments, the method includes administering the composition between 0 days and 2 days after the traumatic pain event. In embodiments, the method includes administering the composition between 0 days and 1 days after the traumatic pain event.

In embodiments, the method includes administering the composition between about 30 days and about 1 days before the traumatic pain event. In embodiments, the method includes administering the composition between about 25 days and about 1 days before the traumatic pain event. In embodiments, the method includes administering the composition between about 20 days and about 1 days before the traumatic pain event. In embodiments, the method includes administering the composition between about 15 days and about 1 days before the traumatic pain event. In embodiments, the method includes administering the composition between about 10 days and about 1 days before the traumatic pain event. In embodiments, the method includes administering the composition between about 9 days and about 1 days before the traumatic pain event. In embodiments, the method includes administering the composition between about 8 days and about 1 days before the traumatic pain event. In embodiments, the method includes administering the composition between about 7 days and about 1 days before the traumatic pain event. In embodiments, the method includes administering the composition between about 6 days and about 1 days before the traumatic pain event. In embodiments, the method includes administering the composition between about 5 days and about 1 days before the traumatic pain event. In embodiments, the method includes administering the composition between about 4 days and about 1 days before the traumatic pain event. In embodiments, the method includes administering the composition between about 3 days and about 1 days before the traumatic pain event. In embodiments, the method includes administering the composition between about 2 days and about 1 days before the traumatic pain event.

In embodiments, the method includes administering the composition between about 30 days and about 2 days before the traumatic pain event. In embodiments, the method includes administering the composition between about 30 days and about 3 days before the traumatic pain event. In embodiments, the method includes administering the composition between about 30 days and about 4 days before the traumatic pain event. In embodiments, the method includes administering the composition between about 30 days and about 5 days before the traumatic pain event. In embodiments, the method includes administering the composition between about 30 days and about 6 days before the traumatic pain event. In embodiments, the method includes administering the composition between about 30 days and about 7 days before the traumatic pain event. In embodiments, the method includes administering the composition between about 30 days and about 8 days before the traumatic pain event. In embodiments, the method includes administering the composition between about 30 days and about 9 days before the traumatic pain event. In embodiments, the method includes administering the composition between about 30 days and about 10 days before the traumatic pain event. In embodiments, the method includes administering the composition between about 30 days and about 15 days before the traumatic pain event. In embodiments, the method includes administering the composition between about 30 days and about 20 days before the traumatic pain event. In embodiments, the method includes administering the composition between about 30 days and about 25 days before the traumatic pain event.

In embodiments, the method includes administering the composition between 30 days and 1 days before the traumatic pain event. In embodiments, the method includes administering the composition between 25 days and 1 days before the traumatic pain event. In embodiments, the method includes administering the composition between 20 days and 1 days before the traumatic pain event. In embodiments, the method includes administering the composition between 15 days and 1 days before the traumatic pain event. In embodiments, the method includes administering the composition between 10 days and 1 days before the traumatic pain event. In embodiments, the method includes administering the composition between about 9 days and 1 days before the traumatic pain event. In embodiments, the method includes administering the composition between about 8 days and about 1 days before the traumatic pain event. In embodiments, the method includes administering the composition between 7 days and 1 days before the traumatic pain event. In embodiments, the method includes administering the composition between 6 days and 1 days before the traumatic pain event. In embodiments, the method includes administering the composition between 5 days and 1 days before the traumatic pain event. In embodiments, the method includes administering the composition between 4 days and 1 days before the traumatic pain event. In embodiments, the method includes administering the composition between 3 days and 1 days before the traumatic pain event. In embodiments, the method includes administering the composition between 2 days and 1 days before the traumatic pain event.

In embodiments, the method includes administering the composition between 30 days and 2 days before the traumatic pain event. In embodiments, the method includes administering the composition between 30 days and 3 days before the traumatic pain event. In embodiments, the method includes administering the composition between 30 days and 4 days before the traumatic pain event. In embodiments, the method includes administering the composition between 30 days and 5 days before the traumatic pain event. In embodiments, the method includes administering the composition between 30 days and 6 days before the traumatic pain event. In embodiments, the method includes administering the composition between 30 days and 7 days before the traumatic pain event. In embodiments, the method includes administering the composition between 30 days and 8 days before the traumatic pain event. In embodiments, the method includes administering the composition between 30 days and 9 days before the traumatic pain event. In embodiments, the method includes administering the composition between 30 days and 10 days before the traumatic pain event. In embodiments, the method includes administering the composition between 30 days and 15 days before the traumatic pain event. In embodiments, the method includes administering the composition between 30 days and 20 days before the traumatic pain event. In embodiments, the method includes administering the composition between 30 days and 25 days before the traumatic pain event.

In embodiments, the method includes administering the composition daily for 30 days before the traumatic pain event. In embodiments, the method includes administering the composition daily for 29 days before the traumatic pain event. In embodiments, the method includes administering the composition daily for 28 days before the traumatic pain event. In embodiments, the method includes administering the composition daily for 27 days before the traumatic pain event. In embodiments, the method includes administering the composition daily for 26 days before the traumatic pain event. In embodiments, the method includes administering the composition daily for 25 days before the traumatic pain event. In embodiments, the method includes administering the composition daily for 24 days before the traumatic pain event. In embodiments, the method includes administering the composition daily for 23 days before the traumatic pain event. In embodiments, the method includes administering the composition daily for 22 days before the traumatic pain event. In embodiments, the method includes administering the composition daily for 21 days before the traumatic pain event. In embodiments, the method includes administering the composition daily for 20 days before the traumatic pain event. In embodiments, the method includes administering the composition daily for 19 days before the traumatic pain event. In embodiments, the method includes administering the composition daily for 18 days before the traumatic pain event. In embodiments, the method includes administering the composition daily for 17 days before the traumatic pain event. In embodiments, the method includes administering the composition daily for 16 days before the traumatic pain event. In embodiments, the method includes administering the composition daily for 15 days before the traumatic pain event. In embodiments, the method includes administering the composition daily for 14 days before the traumatic pain event. In embodiments, the method includes administering the composition daily for 13 days before the traumatic pain event. In embodiments, the method includes administering the composition daily for 12 days before the traumatic pain event. In embodiments, the method includes administering the composition daily for 11 days before the traumatic pain event. In embodiments, the method includes administering the composition daily for 10 days before the traumatic pain event.

In embodiments, the method includes administering the composition twice daily for 30 days before the traumatic pain event. In embodiments, the method includes administering the composition twice daily for 29 days before the traumatic pain event. In embodiments, the method includes administering the composition twice daily for 28 days before the traumatic pain event. In embodiments, the method includes administering the composition twice daily for 27 days before the traumatic pain event. In embodiments, the method includes administering the composition twice daily for 26 days before the traumatic pain event. In embodiments, the method includes administering the composition twice daily for 25 days before the traumatic pain event. In embodiments, the method includes administering the composition twice daily for 24 days before the traumatic pain event. In embodiments, the method includes administering the composition twice daily for 23 days before the traumatic pain event. In embodiments, the method includes administering the composition twice daily for 22 days before the traumatic pain event. In embodiments, the method includes administering the composition twice daily for 21 days before the traumatic pain event. In embodiments, the method includes administering the composition twice daily for 20 days before the traumatic pain event. In embodiments, the method includes administering the composition twice daily for 19 days before the traumatic pain event. In embodiments, the method includes administering the composition twice daily for 18 days before the traumatic pain event. In embodiments, the method includes administering the composition twice daily for 17 days before the traumatic pain event. In embodiments, the method includes administering the composition twice daily for 16 days before the traumatic pain event. In embodiments, the method includes administering the composition twice daily for 15 days before the traumatic pain event. In embodiments, the method includes administering the composition twice daily for 14 days before the traumatic pain event. In embodiments, the method includes administering the composition twice daily for 13 days before the traumatic pain event. In embodiments, the method includes administering the composition twice daily for 12 days before the traumatic pain event. In embodiments, the method includes administering the composition twice daily for 11 days before the traumatic pain event. In embodiments, the method includes administering the composition twice daily for 10 days before the traumatic pain event.

In an aspect the method further includes administering an effective amount of an agent to the subject, wherein the agent is administered between about 1 day and 7 days after a traumatic pain event and wherein said agent is an NAAA (N-Acylethanolamine acid amidase) inhibitor, FAAH (Fatty acid amide hydrolase) inhibitor, PPARα (peroxisome proliferator-activated receptor-α) agonist, PEA (palmitoylethanolamide), acetyl-L-carnitine, α-lipoic acid, or olesoxime. In embodiments, the method includes preventing transition from an acute pain state to a chronic pain state in the subject. In embodiments, the agent is administered between about 24 hours and about 96 hours after the traumatic pain event. In embodiments, the agent is an NAAA (N-Acylethanolamine acid amidase) inhibitor. In embodiments, the agent is a FAAH (Fatty acid amide hydrolase) inhibitor. In embodiments, the agent is a PPARα (peroxisome proliferator-activated receptor-α) agonist. In embodiments, the agent is PEA (palmitoylethanolamide). In embodiments, the agent is acetyl-L-carnitine. In embodiments, the agent is α-lipoic acid. In embodiments, the agent is olesoxime. In embodiments, the agent is administered in a therapeutically effective amount.

In embodiments, the agent is a NAAA inhibitor, FAAH inhibitor, PPARα agonist, or PEA. In embodiments, the agent is a NAAA inhibitor. In embodiments, the agent is a FAAH inhibitor. In embodiments, the agent is a PPARα agonist. In embodiments, the agent is PEA.

In embodiments, the NAAA inhibitor is ARN16186, ARN077, or ARN19702. In embodiments, the NAAA inhibitor is ARN16186. In embodiments, the NAAA inhibitor is ARN077. In embodiments, the NAAA inhibitor is ARN19702. In embodiments, the NAAA inhibitor is described in J Med Chem. 2020 Jul. 23; 63(14):7475-7490, which is incorporated herein by reference in its entirety and for all purposes. In embodiments, the NAAA inhibitor is described in WO 2013/078430, US 2013/0281490, WO 2009/049238, US 2014/0094508, WO 2014/144836, US 2016/0068482, WO 2017/201103, or US 2019/0177313, which are incorporated herein by reference in their entirety and for all purposes.

In embodiments, the FAAH inhibitor is URB597, URB937, an analog of URB 597, or an analog of URB 937. In embodiments, the FAAH inhibitor is URB597. In embodiments, the FAAH inhibitor is URB937. In embodiments, the FAAH inhibitor is an analog of URB 597. In embodiments, the FAAH inhibitor is an analog of URB 937. In embodiments, the FAAH inhibitor is described in J Med Chem. 2017 Jan. 12; 60(1):4-46, which is incorporated herein by reference in its entirety and for all purposes. In embodiments, the FAAH inhibitor is described in WO 2015/157313, US 2017/0088510, WO 2012/015704, US 2013/0217764, WO 2013/028570, or US 2014/0288170, which are incorporated herein by reference in their entirety and for all purposes.

In embodiments, the PPARα agonist is natural or unnatural PPARα agonist.

In embodiments, the PPARα agonist is GW7647, PEA (palmitoylethanolamide), or OEA (oleoylethanolamide). In embodiments, the PPARα agonist is GW7647. In embodiments, the PPARα agonist is PEA (palmitoylethanolamide). In embodiments, the PPARα agonist is OEA (oleoylethanolamide). In embodiments, the PPARα agonist is described in Expert Opin Investig Drugs. 2017 May; 26(5):593-60, which is incorporated by reference, herein, in its entirety.

In embodiments, the method further includes administering the agent between about 8 days and 30 days after the traumatic pain event. In embodiments, the method further includes administering the agent between about 8 days and 10 days after the traumatic pain event. In embodiments, the method further includes administering the agent between about 8 days and 14 days after the traumatic pain event. In embodiments, the method further includes administering the agent between about 8 days and 21 days after the traumatic pain event. In embodiments, the method further includes administering the agent between about 8 days and 28 days after the traumatic pain event. In embodiments, the method further includes administering the agent between about 10 days and 14 days after the traumatic pain event. In embodiments, the method further includes administering the agent between about 10 days and 21 days after the traumatic pain event. In embodiments, the method further includes administering the agent between about 10 days and 28 days after the traumatic pain event. In embodiments, the method further includes administering the agent between about 15 days and 20 days after the traumatic pain event. In embodiments, the method further includes administering the agent between about 15 days and 30 days after the traumatic pain event.

In embodiments, the method further includes continually administering the agent between about 8 days and 30 days after the traumatic pain event. In embodiments, the method further includes continually administering the agent between about 8 days and 10 days after the traumatic pain event. In embodiments, the method further includes continually administering the agent between about 8 days and 14 days after the traumatic pain event. In embodiments, the method further includes continually administering the agent between about 8 days and 21 days after the traumatic pain event. In embodiments, the method further includes continually administering the agent between about 8 days and 28 days after the traumatic pain event. In embodiments, the method further includes continually administering the agent between about 10 days and 14 days after the traumatic pain event. In embodiments, the method further includes continually administering the agent between about 10 days and 21 days after the traumatic pain event. In embodiments, the method further includes continually administering the agent between about 10 days and 28 days after the traumatic pain event. In embodiments, the method further includes continually administering the agent between about 15 days and 20 days after the traumatic pain event. In embodiments, the method further includes continually administering the agent between about 15 days and 30 days after the traumatic pain event.

In embodiments, the traumatic pain event is due to accidental physical injury, invasive surgery, or acute illness. In embodiments, the traumatic pain event is due to accidental physical injury. In embodiments, the traumatic pain event is due to invasive surgery. In embodiments, the traumatic pain event is due to acute illness.

In embodiments, the acute physical injury is a concussion, in bone fracture, or an internal injury. In embodiments, the acute physical injury is a concussion. In embodiments, the acute physical injury is a bone fracture. In embodiments, the acute physical injury is an internal injury.

In embodiments, the invasive surgery is cardiac, breast surgery, or orthopedic surgery. In embodiments, the invasive surgery is cardiac surgery. In embodiments, the invasive surgery is breast surgery. In embodiments, the invasive surgery is orthopedic surgery. In embodiments, the invasive surgery is knee arthroplasty, hip replacement surgery, mastectomy, open-heart surgery, hernia repair, thoracotomy, caesarian section, amputation, or open cholecystoctomy. In embodiments, the invasive surgery is knee arthroplasty. In embodiments, the invasive surgery is hip replacement surgery. In embodiments, the invasive surgery is mastectomy. In embodiments, the invasive surgery is open-heart surgery. In embodiments, the invasive surgery is hernia repair. In embodiments, the invasive surgery is thoracotomy. In embodiments, the invasive surgery is caesarian section. In embodiments, the invasive surgery is amputation. In embodiments, the invasive surgery is open cholecystoctomy.

In embodiments, the agent is administered perioperatively. In embodiments, the agent is administered during the period of time before, during, or after surgery. The term “perioperative” or “perioperatively” is used in accordance with its plain ordinary meaning and generally refers to the time period approximately prior to, during, and/or approximately after a surgical operation. For example, “perioperative” or “perioperatively” may refer to the period of time extending from patient arrival (e.g., admission) at a surgical facility (e.g., hospital, clinic, doctor's office) for a surgical operation until the time the patient leaves (e.g., is discharged from) the surgical operation. In embodiments, perioperative includes surgical facility admission, anesthesia, surgery, and recovery from the surgical operation.

In an aspect is provided a method of preventing chronic pain in a subject, the method including administering perioperatively an effective amount of an agent to the subject, wherein the agent is a NAAA inhibitor, FAAH inhibitor, PPARα agonist, PEA, acetyl-L-carnitine, α-lipoic acid, or olesoxime. In embodiments, the NAAA inhibitor is as described herein, including in embodiments. In embodiments, the FAAH inhibitor is as described herein, including in embodiments. In embodiments, the PPARα agonist is as described herein, including in embodiments.

In an aspect is provided a method of preventing peripheral neuropathic pain in a subject previously treated with an anti-cancer agent, the method including administering an effective amount of a composition to said patient, said composition comprising at least one purified free amino acid, at least one purified fatty acid, and purified fatty acid amide, wherein chronic pain is prevented in the subject.

In an aspect is provided a method of preventing peripheral neuropathic pain in a subject previously treated with an anti-cancer agent, the method including administering an effective amount of a composition to said patient, said compositions comprising purified palmitoylethanolamide (PEA), at least one purified free amino acid and at least one purified fatty acid, wherein chronic pain is prevented in the subject.

In an aspect the method further comprises administering an effective amount of an agent to the cancer patient, wherein said agent is a NAAA inhibitor, FAAH inhibitor, PPARα agonist, PEA, acetyl-L-carnitine, α-lipoic acid, or olesoxime. In embodiments, the NAAA inhibitor is as described herein, including in embodiments. In embodiments, the FAAH inhibitor is as described herein, including in embodiments. In embodiments, the PPARα agonist is as described herein, including in embodiments.

In embodiments, the method further includes administering an anti-cancer agent (e.g., as described herein). In embodiments, the chronic pain is caused by the anti-cancer agent. In embodiments, the chronic pain is peripheral neuropathy. In embodiments, the chronic pain is allodynia. In embodiments, the chronic pain is hyperalgesia. In embodiments, the chronic pain is paresthesia. The term “paresthesia” is used in accordance with its plain ordinary meaning and generally refers to an abnormal sensation, e.g., tingling or pricking. In embodiments, the chronic pain is numbing.

In an aspect is provided a method of preventing chronic pain in a diabetic patient, the method including administering an effective amount of a composition to said patient, said compositions comprising at least one purified free amino acid, at least one purified fatty acid, and purified fatty acid amide, wherein chronic pain is prevented in the subject.

In an aspect is provided a method of preventing chronic pain in a diabetic patient, the method including administering an effective amount of a composition to said patient, said compositions comprising purified palmitoylethanolamide (PEA), at least one purified free amino acid and at least one purified fatty acid, wherein chronic pain is prevented in the subject.

In an aspect the method further includes administering an effective amount of an agent to the diabetic patient, wherein said agent is a NAAA inhibitor, FAAH inhibitor, PPARα agonist, PEA, acetyl-L-carnitine, α-lipoic acid, or olesoxime. In embodiments, the NAAA inhibitor is as described herein, including in embodiments. In embodiments, the FAAH inhibitor is as described herein, including in embodiments. In embodiments, the PPARα agonist is as described herein, including in embodiments.

In embodiments, the chronic pain is chronic peripheral neuropathy. In embodiments, the chronic pain is chronic polyneuropathy. In embodiments, the chronic pain is hyperalgesia. In embodiments, the chronic pain is paresthesia (e.g., tingling or pricking). In embodiments, the chronic pain is numbing.

In embodiments, the agent is not morphine. In embodiments, the agent is not gabapentin. In embodiments, the agent is not ketamine. In embodiments, the agent is not ketoprofen.

In embodiments, the method further includes administering an opioid analgesic for acute postoperative pain. In embodiments, the opioid analgesic is morphine and the composition enhances the analgesic effects of morphine.

The methods described herein can result in reduction of postoperative opioid consumption, reduction in the length of hospital admission after surgery, decreased preoperative anxiety, and fewer sedative medication requests.

### IV. Kits

In an aspect is provided kits comprising any of the compositions or compounds described above. In aspects the compositions and compounds are combined. In aspects, the kit further includes any of the agents described above. In aspects, one or more of the compounds or agents are in a separate container. The kits can further include instructions for preparation and use.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

These examples are provided for illustrative purposes only and not to limit the scope of the claims provided herein.

### Example 1: Effects of Diet on Chronic Pain In Vivo

Applicants found that formalin produces in mice an ‘acute’ nocifensive reaction that is followed by localized inflammation and persistent neuropathological state whose multimodal manifestations are strikingly reminiscent of human chronic pain. Without wishing to be held by theory, Applicants discovered a critical period for the transition from acute pain to chronic pain, which briefly opens following tissue damage and coincides with massive metabolic changes in local spinal cord. These changes include decreased concentrations of free amino acids (alanine, threonine, proline, serine, leucine, isoleucine, valine, phenylalanine, tyrosine, methionine, cysteine, tryptophan), nonesterified unsaturated fatty acids (oleic acid, erucic acid) and the fatty acid amide palmitoylethanolamide (PEA). A study to test whether providing animals with these missing compounds through an enriched diet abrogates pain chronification in formalin-injected mice was therefore performed. Three distinct experimental diets (EDs) were tested in the formalin test in mice. The EDs utilized here were a modified version of the standard rodent diet (STD) enriched with either free amino acids plus fatty acids (ED-1), PEA (ED-2), or the combination of both (COMBO). The results show that formalin injection elicits in the injected paw profound swelling associated with persistent bilateral hypersensitivity to slightly painful heat stimuli (heat hyperalgesia) and to normally innocuous mechanical stimuli (mechanical allodynia). Pain hypersensitivity was accompanied by emotional and cognitive disturbances. Dietary intervention with either ED-1 or ED-2 attenuated formalin-evoked sensory hypersensitivity, yet failed to stop the emergence of persistent painful states upon withdrawal of EDs replaced by STD. By contrast, the combination of both halted chronic pain development, normalizing sensory abnormalities, anxiety-like behaviors and memory deficits. Consequently, dietary enrichment with the combination of free amino acids, fatty acids, and PEA abrogates pain chronification following surgical or accidental tissue injury.

### Example 2: Materials and Methods

### Animals

Briefly CD-1 mice (7 weeks of age upon arrival; Charles River, Wilmington, MA) were maintained in a pathogen-free environment on 12-hour light/dark cycle at controlled temperature (22° C.) and humidity (50 to 60%). Food and water were available ad libitum. Animals were randomly assigned to study groups and, before the start of the experiments, were handled for three consecutive days (˜3 min per animal/day), and behavioral testing was conducted during the light phase of the light/dark cycle. Efforts were made to minimize the number of animals used and their discomfort. The study complied with all ethical regulations of the National Institutes of Health (NIH) Guide for the Care and Use of Laboratory Animals and the recommendation of the International Association for the Study of Pain. Experimental procedures were approved by the Animal Care and Use Committee of the University of California, Irvine (AUP-20-117).

### Dietary Intervention

Mice were randomly assigned to receive either standard rodent diet (SD) or experimental diets (EDs: ED-1, ED-2 or COMBO) for a period of 3 weeks. Then, the animals were challenged with formalin (1% vol, intraplantar injection) or saline injections (Sham) and kept to their respective diets for a week. In separate experiments, the animals were subjected to sham or spared nerve injury (SNI) of their sciatic nerve and kept to their respective diets for two weeks. Nociceptive behaviors, paw edema, cognitive deficits (novel object recognition test, NOR) and emotional disturbances (elevated plus maze test, EPM) produced by formalin injections or SNI were evaluated on various time points after the challenge.

### Diet Composition

The experimental diets (EDs) were a modified version of the standard rodent chow (SD; Teklad Diets, Madison, WI, USA) enriched with either free amino acids plus fatty acids (ED1), or palmitoylethanolamide (PEA) (ED2) or combination of free amino acids, fatty acids and PEA (COMBO). Mice were fed ad libitum with their respective diets.

TABLE 3
components of ED1

| | Animal dose |
|---|---|
| Active ingredients | in mg/kg |
| Oleic acid | 6200 |
| Erucic acid | 310 |
| Alanine | 2362.5 |
| Threonine | 1625 |
| Proline | 3000 |
| Serine | 1250 |
| Leucine | 3875 |
| Isoleucine | 3062.5 |
| Valine | 2875 |
| Phenylalanine | 1875 |
| Tyrosine | 1125 |
| Methionine | 750 |
| Cysteine | 562.5 |
| Tryptophan | 762.5 |

TABLE 4
components of ED2

| | Animal dose |
|---|---|
| Active ingredients | in mg/kg |
| PEA | 103.75 |

TABLE 5
components of Combo

| | Animal dose |
|---|---|
| Active ingredients | in mg/kg |
| PEA | 103.75 |
| Oleic acid | 6200 |
| Erucic acid | 310 |
| Alanine | 2362.5 |
| Threonine | 1625 |
| Proline | 3000 |
| Serine | 1250 |
| Leucine | 3875 |
| Isoleucine | 3062.5 |
| Valine | 2875 |
| Phenylalanine | 1875 |
| Tyrosine | 1125 |
| Methionine | 750 |
| Cysteine | 562.5 |
| Tryptophan | 762.5 |

### Behavioral Tests

### Formalin

Mice were injected formalin (1% vol, 20 μl) or saline into the plantar surface of the right hind paw, as reported previously (Mabou Tagne et al., 2021). Following injection, the mice were immediately transferred to a transparent observation chamber where nocifensive behavior (time spent licking or biting the injected paw and number of paw shakings) was videorecorded for 60 min and quantified by blinded observer. Mechanical allodynia, heat hyperalgesia, and paw edema were measured on post-formalin day (PFD) 7 and 14 in both injected and noninjected paws.

### Spared Nerve Injury

To determine spared nerve injury, a protocol described previously in (Guida et al., 2020) was used. The mice were anesthetized with isoflurane, and the right common sciatic nerve was exposed by blunt dissection at the level of its trifurcation into sural, tibial, and common peroneal nerves under aseptic conditions. Then, the common peroneal and the tibial nerves were tightly-ligated (nonabsorbable 6.0 silk for mouse) and sectioned distal to the ligation, removing 2-4 mm of the distal nerve stump. The sural nerve was left intact. The wound was closed with a single muscle suture and skin clips. In sham-operated animals, the sciatic nerve was exposed but not transected.

### Mechanical Allodynia

Mechanical allodynia was assessed using a dynamic plantar aesthesiometer (Ugo Basile, Comerio, Italy). After a 45-min habituation period in transparent cages positioned on a wire mesh surface, a mechanical stimulus was applied to the plantar surface of both hind paws by an automated steel filament exerting an increasing force ranging from 0 to 5 g over 10 s. Three withdrawal thresholds (in grams) were recorded and averaged.

### Paw Edema

Paw edema was measured with a digital caliper (Fisher Scientific, USA) and is expressed as the difference (Δpaw thickness, mm) between ipsilateral and contralateral paws.

### Sensitivity to Heat

Sensitivity to heat was measured using a Hargreaves plantar test apparatus (San Diego Instruments, San Diego, USA). After a 45-min habituation period, the plantar surface of both hind paws was exposed to a beam of radiant heat through the glass floor. The cutoff time was set at 15 s. The stimulation was repeated three times with an interval of 2 min between stimuli, and latencies (in second) to withdraw the paw were recorded and averaged.

### Elevated Plus Maze Test

Each mouse was placed in the central platform of the maze, facing the open arm opposite to the experimenter, and behavior was recorded using the Debut video capture software (NCH Software, Canberra, Australia). A blinded observer measured the amount of time spent in the open and closed arms, as well as the number of open and closed arm entries. The open arms of the maze were illuminated at 150 to 170 lux, and the closed arms at 40 to 50 lux. The anxiety index was calculated as follows

$$Anxiety index = 1 - \frac{( \frac{time in open arms}{total time} ) + ( \frac{n umber of open arms entries}{total entries} )}{2}$$

### Novel Object Recognition Test

The test was conducted over 3 days. On day 1, mice were habituated to the empty arena for 20 min. On day 2, they were returned to the arena, which now contained two identical objects. On day 3, one of the objects was replaced with another one of different shape, color, and texture. Mice were allowed to explore the arena for 10 min, and the total time spent exploring each object (i.e., nosing and sniffing at a distance≤2 cm) was recorded by a blinded observer. The discriminatory index (D.I.) was calculated as follows:

$$D . I . = \frac{\begin{matrix}( time of novel object exploration ) + \\ ( time of familiar object exploration )\end{matrix}}{total exploration time}$$

### Example 3: Effects of Diet on Spinal Cord Energy Crisis and Metabolite Levels

This example describes the determination of metabolic changes in the mouse spinal cord after formalin-induced transition to pain chronicity and treatment with a medical food.

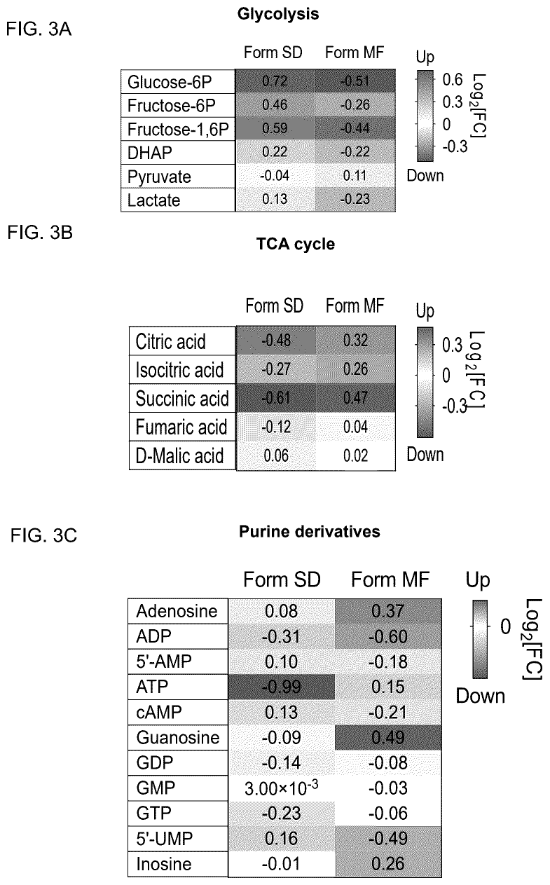

Briefly, mice were maintained on a standard diet (SD) or on medical food (MF) compositions as described in Example 2 and then treated after three weeks with formalin as described in Example 1. Samples of the mouse spinal cords were taken at 24 h, 48h, and 72 hrs. after formalin treatment and subjected to metabolomics analysis. The levels of metabolites involved in glycolysis, TCA cycle, purine derivatives, amino acids, urea cycle, acetylated amino acids, fatty acids, and phospholipids were measured (FIG. 3).

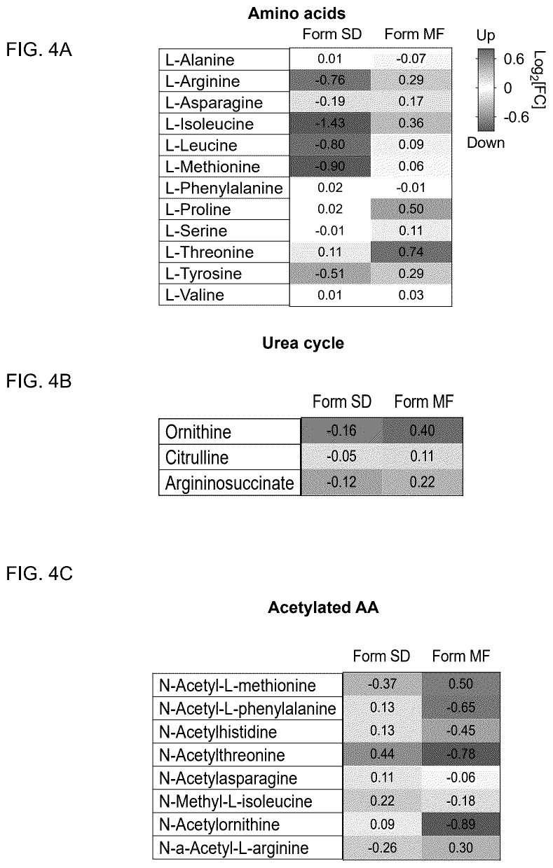

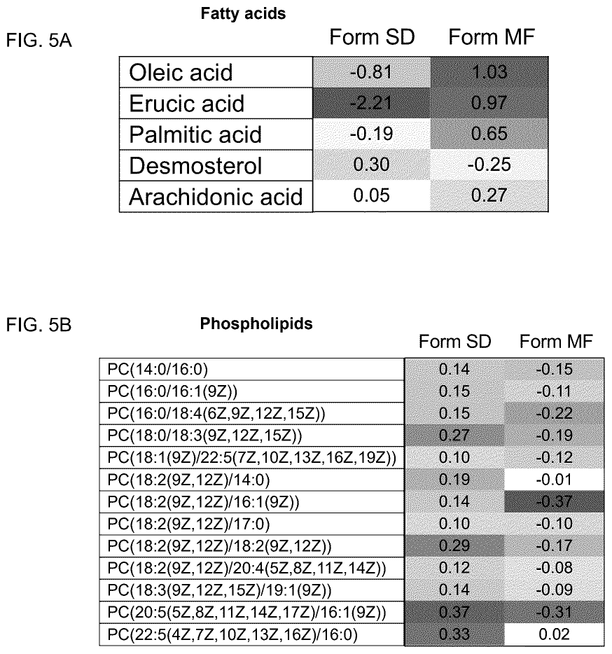

The results show, that in mice treated with the medical food metabolic changes were lessened or reversed. The metabolic shift from the TCA cycle to aerobic glycolysis (FIGS. 3A and 3B), which occurs during the transition to chronic pain, is prevented by the medical food. The energy crisis (low ATP, FIG. 3C) is also blocked. Levels of amino acids (FIG. 4A) and urea cycle intermediates (FIG. 4B), which drop during the transition to chronic pain, are normalized by the medical food. Changes in acetylated amino acid levels are countered (FIG. 4C). Levels of fatty acids (FIG. 5A) and phospholipids (FIG. 5B), which are differentially altered during the transition to chronic pain, are normalized by the medical food.

### Example 4: Effects of Diet in a Model of Hyperalgesic Priming

This example describes the determination of effects of diet in mice after hyperalgesic priming and treatment with a medical food.

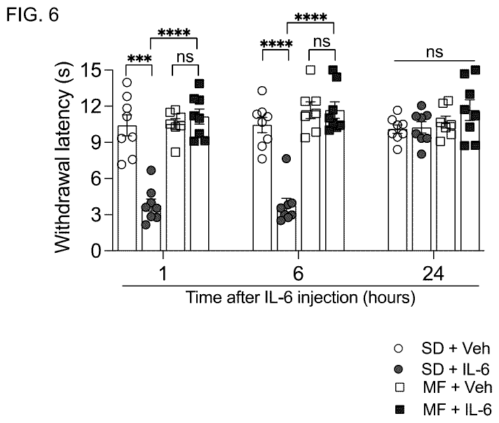

Briefly, male mice were maintained on a standard diet (SD) or on medical food (MF) compositions as described in Example 2 and treated after three weeks with IL-6 (5 ng, intraplantar) or its vehicle. IL-6-primed mice were challenged with the pain-inducing compound, prostaglandin E₂, and then subjected to behavioral tests to measure pain hypersensitivity (hyperalgesia). The results show that administration of interleukin-6 (IL-6) in male mice fed a standard diet (SD) causes local heat hypersensitivity (assessed as withdrawal latency in seconds) that lasts>6h (FIG. 6). The response is absent in mice fed the medical food (MF) (FIG. 6). The results show that the medical food suppresses the acute hyperalgesia caused by IL-6.

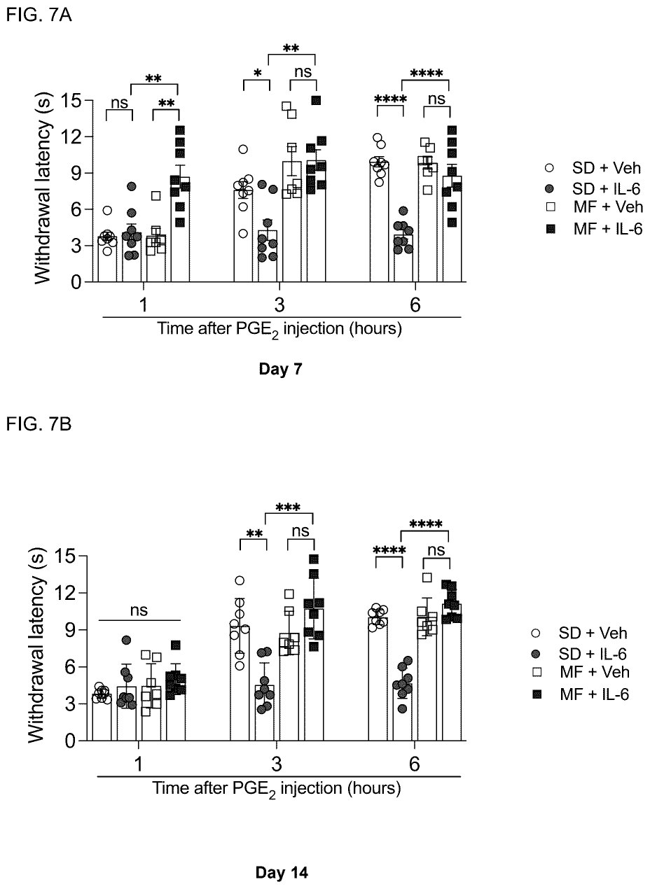

On day 7 and day 14 after IL-6 injection, mice were additionally injected with prostaglandin E₂ (PGE₂, 100 ng, intraplantar). The results show that administration of prostaglandin E₂ in male mice primed with IL-6 causes local heat hypersensitivity in animals fed a standard diet (SD) but not in those fed the medical food (MF) (FIGS. 7A and 7B). Together, the results suggest that the MF prevents the induction of inflammatory hyperalgesia and may be effective in alleviating post-surgical pain in addition to preventing the transition to pain chronicity.

### Example 5: Effects of Diet on Morphine Antinociception

This example describes the determination of effects of diet in mice after morphine-induced antinociception and treatment with a medical food.

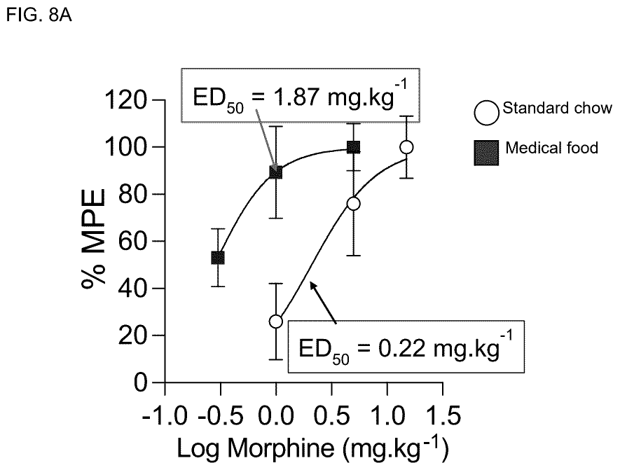

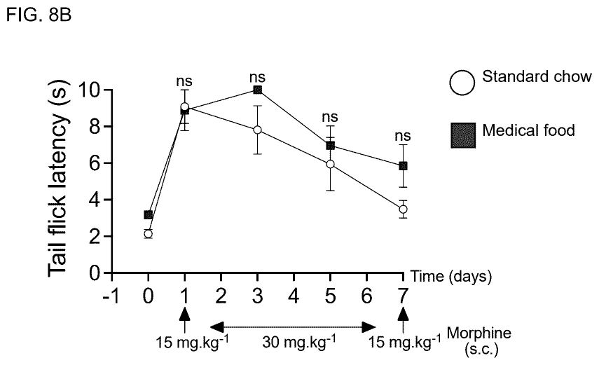

Briefly, morphine (mg/kg, subcutaneous) was administered in animals fed a standard diet or the medical food. The response is presented as percentage of Maximum Possible Effect (MPE) (FIG. 8A). Also included are median affective dose (ED₅₀) values to produce antinociception in the two groups of mice. The results suggest that the MF enhances the analgesic effects of morphine, which is frequently used to treat post-surgical pain.

Additionally, effects of the medical food on tolerance to the antinociceptive effects of morphine in male mice were measured. The antinociceptive effects of morphine (15 mg/kg, subcutaneous) were assessed on day 1. Tolerance was induced by administering morphine (30 mg/kg) on days 2-6. Tolerance was assessed on day 7 by injecting 15 mg/kg morphine (FIG. 8B). The results suggest that the MF does not affect morphine-induced tolerance, which can curtail post-surgical analgesia.

### REFERENCES

Guida, F., De Gregorio, D., Palazzo, E., Ricciardi, F., Boccella, S., Belardo, C., Iannotta, M., Infantino, R., Formato, F., Marabese, I., Luongo, L., de Novellis, V., & Maione, S. (2020). Behavioral, biochemical and electrophysiological changes in spared nerve injury model of neuropathic pain. International Journal of Molecular Sciences, 21(9), 1-21. https://doi.org/10.3390/ijms21093396
Mabou Tagne, A., Fotio, Y., Lin, L., Squire, E., Ahmed, F., Rashid, T. I., Karimian Azari, E., & Piomelli, D. (2021). Palmitoylethanolamide and hemp oil extract exert synergistic anti-nociceptive effects in mouse models of acute and chronic pain. Pharmacological Research, 167, 105545. https://doi.org/10.1016/j.phrs.2021.105545

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### P EMBODIMENTS

P Embodiment 1. A method of preventing chronic pain in a subject after a traumatic pain event, the method comprising administering an effective amount of a composition to the subject, wherein the composition comprises at least one purified free amino acid, at least one purified fatty acid, and purified fatty acid amide wherein chronic pain is prevented in the subject.

P Embodiment 2. The method of embodiment 1, wherein the fatty acid amide is purified palmitoylethanolamide (PEA).

P Embodiment 3. The method of embodiment 1 or 2, wherein the composition is administered orally.

P Embodiment 4. The method of embodiment 1 or 3, wherein the purified fatty acid is oleic acid or erucic acid.

P Embodiment 5. The method of embodiment 4, wherein the subject is a human and the dosage range of oleic acid is between 48 to 496 mg/kg.

P Embodiment 6. The method of embodiment 4 or 5, wherein the subject is a human and the dosage range of erucic acid is 2 to 25 mg/kg.

P Embodiment 7. The method of any of embodiments 1 to 6, wherein the purified free amino acid is alanine, threonine, proline, serine, leucine, isoleucine, valine, phenylalanine, tyrosine, methionine, cysteine, or tryptophan.

P Embodiment 8. The method of embodiment 7, wherein the subject is human and the dosage range of alanine is 20 to 189 mg/kg.

P Embodiment 9. The method of embodiment 7 or 8, wherein the subject is human and the dosage range of threonine is 12 to 130 mg/kg.

P Embodiment 10. The method of any of embodiments 7 to 9, wherein the subject is human and the dosage range of proline is 24 to 240 mg/kg.

P Embodiment 11. The method of any of embodiments 7 to 10, wherein the subject is human and the dosage range of serine is 10 to 100 mg/kg.

P Embodiment 12. The method of any of embodiments 7 to 11, wherein the subject is human and the dosage range of leucine is 28 to 310 mg/kg.

P Embodiment 13. The method of any of embodiments 7 to 12, wherein the subject is human and the dosage range of isoleucine is 24 to 245 mg/kg.

P Embodiment 14. The method of any of embodiments 7 to 13, wherein the subject is human and the dosage range of valine is 22 to 230 mg/kg.

P Embodiment 15. The method of any of embodiments 7 to 14, wherein the subject is human and the dosage range of phenylalanine is 15 to 150/kg.

P Embodiment 16. The method of any of embodiments 7 to 15, wherein the subject is human and the dosage range of tyrosine is 9 to 90 mg/kg.

P Embodiment 17. The method of any of embodiments 7 to 16, wherein the subject is human and the dosage range of methionine is 6 to 60 mg/kg.

P Embodiment 18. The method of any of embodiments 7 to 17, wherein the subject is human and the dosage range of cysteine is 4 to 45 mg/kg.

P Embodiment 19. The method of any of embodiments 7 to 18, wherein the subject is human and the dosage range of tryptophan is 6 to 61 mg/kg.

P Embodiment 20. The method of any of embodiments 1 to 19, wherein the subject is human and the dosage range of PEA is 0.8 to 9 mg/kg.

P Embodiment 21. The method of any of embodiments 1 to 20, wherein the composition is administered between about 0 days and about 30 days after the traumatic pain event.

P Embodiment 22. The method of any of embodiments 1 to 21, wherein the composition is administered between about 30 days and about 1 day before the traumatic pain event.

P Embodiment 23. The method of any of embodiments 1 to 22, wherein the traumatic pain event is due to physical injury, invasive surgery, or acute illness.

P Embodiment 24. The method of any of embodiments 1 to 23, wherein the physical injury is accidental physical injury.

P Embodiment 25. The method of embodiment 23, wherein the physical injury is acute physical injury.

P Embodiment 26. The method of embodiment 25, wherein the acute physical injury is a concussion, a bone fracture, or an internal injury.

P Embodiment 27. The method of embodiment 23, wherein the invasive surgery is knee arthroplasty, hip replacement surgery, mastectomy, open-heart surgery, hernia repair, thoracotomy, caesarian section, amputation, or open cholecystoctomy.

P Embodiment 28. The method of embodiment 27, wherein the composition is administered perioperatively.

P Embodiment 29. The method of any of embodiments 1 to 28, further comprising administering an agent between about 1 day and 7 days after the traumatic pain event and wherein the agent is a NAAA inhibitor, FAAH inhibitor, PPARα agonist, PEA, acetyl-L-carnitine, α-lipoic acid, or olesoxime.

P Embodiment 30. The method of embodiment 29, further comprising administering the agent between about 8 days and 30 days after the traumatic pain event.

P Embodiment 31. The method of embodiment 29, further comprising continually administering the agent between about 8 days and 30 days after the traumatic pain event.

P Embodiment 32. A method of preventing chronic pain in a subject after a traumatic pain event, the method comprising administering an effective amount of a composition to the subject, wherein the composition comprises at least one purified free amino acid, at least one purified fatty acid, and purified fatty acid amide wherein chronic pain is prevented in the subject.

P Embodiment 33. The method of embodiment 32, wherein the fatty acid amide is purified palmitoylethanolamide (PEA).

P Embodiment 34. A method of preventing peripheral neuropathic pain in a subject previously treated with an anti-cancer agent, the method comprising administering an effective amount of a composition to the subject, wherein the composition comprises at least one purified free amino acid, at least one purified fatty acid, and purified fatty acid amide wherein chronic pain is prevented in the subject.

P Embodiment 35. The method of embodiment 34, wherein the fatty acid amide is purified palmitoylethanolamide (PEA).

P Embodiment 36. The method of embodiment 32 or 34, wherein the composition is administered orally.

P Embodiment 37. The method of embodiment 32 or 36, wherein the purified fatty acid is oleic acid or erucic acid.

P Embodiment 38. The method of embodiment 37, wherein the subject is a human and the dosage range of oleic acid is between 48-496 mg/kg.

P Embodiment 39. The method of embodiment 37 or 38, wherein the subject is a human and the dosage range of erucic acid is 2 to 25 mg/kg.

P Embodiment 40. The method of any of embodiments 32 to 39, wherein the purified free amino acid is alanine, threonine, proline, serine, leucine, isoleucine, valine, phenylalanine, tyrosine, methionine, cysteine, or tryptophan.

P Embodiment 41. The method of embodiment 40, wherein the subject is human and the dosage range of alanine is 20 to 189 mg/kg.

P Embodiment 42. The method of embodiment 40 or 41, wherein the subject is human and the dosage range of threonine is 12 to 130 mg/kg.

P Embodiment 43. The method of any of embodiments 40 to 42, wherein the subject is human and the dosage range of proline is 24 to 240 mg/kg.

P Embodiment 44. The method of any of embodiments 40 to 43, wherein the subject is human and the dosage range of serine is 10 to 100 mg/kg.

P Embodiment 45. The method of any of embodiments 40 to 44, wherein the subject is human and the dosage range of leucine is 28 to 310 mg/kg.

P Embodiment 46. The method of any of embodiments 40 to 45, wherein the subject is human and the dosage range of isoleucine is 24 to 245 mg/kg.

P Embodiment 47. The method of any of embodiments 40 to 46, wherein the subject is human and the dosage range of valine is 22 to 230 mg/kg.

P Embodiment 48. The method of any of embodiments 40 to 47, wherein the subject is human and the dosage range of phenylalanine is 15 to 150/kg.

P Embodiment 49. The method of any of embodiments 40 to 48, wherein the subject is human and the dosage range of tyrosine is 9 to 90 mg/kg.

P Embodiment 50. The method of any of embodiments 40 to 49, wherein the subject is human and the dosage range of methionine is 6 to 60 mg/kg.

P Embodiment 51. The method of any of embodiments 40 to 50, wherein the subject is human and the dosage range of cysteine is 4 to 45 mg/kg.

P Embodiment 52. The method of any of embodiments 40 to 51, wherein the subject is human and the dosage range of tryptophan is 6 to 61 mg/kg.

P Embodiment 53. The method of any of embodiments 32 to 52, wherein the subject is human and the dosage range of PEA is 0.8 to 9 mg/kg.

P Embodiment 54. The method of any of embodiments 32 to 53, wherein the subject has or previously had cancer.

P Embodiment 55. The method of any of embodiments 32 to 54, further comprising administering an anti-cancer agent.

P Embodiment 56. The method of embodiment 55, wherein the chronic pain is caused by the anti-cancer agent.

P Embodiment 57. The method of any of embodiments 32 to 56, wherein the chronic pain is chronic peripheral neuropathy.

P Embodiment 58. The method of any of embodiments 32 to 56, wherein the chronic pain is allodynia.

P Embodiment 59. A method of preventing chronic pain in a subject with diabetes, the method comprising administering an effective amount of a composition to the subject, wherein the composition comprises at least one purified free amino acid, at least one purified fatty acid, and purified fatty acid amide wherein chronic pain is prevented in the subject.

P Embodiment 60. The method of embodiment 59, wherein the fatty acid amide is purified palmitoylethanolamide (PEA).

P Embodiment 61. The method of embodiment 59, wherein the composition is administered orally.

P Embodiment 62. The method of embodiment 59 or 61, wherein the purified fatty acid is oleic acid or erucic acid.

P Embodiment 63. The method of embodiment 62, wherein the subject is a human and the dosage range of oleic acid is between 48-496 mg/kg.

P Embodiment 64. The method of embodiment 62 or 63, wherein the subject is a human and the dosage range of erucic acid is 2 to 25 mg/kg.

P Embodiment 65. The method of any of embodiments 59 to 64, wherein the purified free amino acid is alanine, threonine, proline, serine, leucine, isoleucine, valine, phenylalanine, tyrosine, methionine, cysteine, or tryptophan.

P Embodiment 66. The method of embodiment 65, wherein the subject is human and the dosage range of alanine is 20 to 189 mg/kg.

P Embodiment 67. The method of embodiment 65 or 66, wherein the subject is human and the dosage range of threonine is 12 to 130 mg/kg.

P Embodiment 68. The method of any of embodiments 65 to 67, wherein the subject is human and the dosage range of proline is 24 to 240 mg/kg.

P Embodiment 69. The method of any of embodiments 65 to 68, wherein the subject is human and the dosage range of serine is 10 to 100 mg/kg.

P Embodiment 70. The method of any of embodiments 65 to 69, wherein the subject is human and the dosage range of leucine is 28 to 310 mg/kg.

P Embodiment 71. The method of any of embodiments 65 to 70, wherein the subject is human and the dosage range of isoleucine is 24 to 245 mg/kg.

P Embodiment 72. The method of any of embodiments 65 to 71, wherein the subject is human and the dosage range of valine is 22 to 230 mg/kg.

P Embodiment 73. The method of any of embodiments 65 to 72, wherein the subject is human and the dosage range of phenylalanine is 15 to 150/kg.

P Embodiment 74. The method of any of embodiments 65 to 73, wherein the subject is human and the dosage range of tyrosine is 9 to 90 mg/kg.

P Embodiment 75. The method of any of embodiments 65 to 74, wherein the subject is human and the dosage range of methionine is 6 to 60 mg/kg.

P Embodiment 76. The method of any of embodiments 65 to 75, wherein the subject is human and the dosage range of cysteine is 4 to 45 mg/kg.

P Embodiment 77. The method of any of embodiments 65 to 76, wherein the subject is human and the dosage range of tryptophan is 6 to 61 mg/kg.

P Embodiment 78. The method of any of embodiments 59 to 77, wherein the subject is human and the dosage range of PEA is 0.8 to 9 mg/kg.

P Embodiment 79. The method of any of embodiments 59 to 78, wherein the chronic pain is chronic peripheral neuropathy.

P Embodiment 80. The method of any of embodiments 59 to 78, wherein the chronic peripheral neuropathy is chronic polyneuropathy.

P Embodiment 81. The method of any of embodiments 1 to 80, further comprising administering an effective amount of an agent to the subject, wherein said agent is administered between about 1 day and 7 days after a traumatic pain event and wherein said agent is a NAAA inhibitor, FAAH inhibitor, PPARα agonist, acetyl-L-carnitine, α-lipoic acid, or olesoxime.

P Embodiment 82. The method of embodiment 81, further comprising administering said agent between about 8 days and 30 days after the traumatic pain event.

P Embodiment 83. The method of embodiment 81, further comprising continually administering said agent between about 8 days and 30 days after the traumatic pain event.

P Embodiment 84. The method of one of embodiments 81 to 83, wherein the agent is a NAAA inhibitor, a FAAH inhibitor, or a PPARα agonist.

P Embodiment 85. The method of one of embodiments 81 to 84, wherein the NAAA inhibitor is ARN16186, ARN077, or ARN19702.

P Embodiment 86. The method of one of embodiments 81 to 84, wherein the FAAH inhibitor is URB597 or an analog of URB 597.

P Embodiment 87. The method of one of embodiments 81 to 84, wherein the FAAH inhibitor is URB937 or an analog of URB 937.

P Embodiment 88. The method of one of embodiments 81 to 84, wherein the PPARα agonist is a natural PPARα agonist.

P Embodiment 89. The method of one of embodiments 81 to 84, wherein the PPARα agonist is an unnatural PPARα agonist.

P Embodiment 90. The method of one of embodiments 81 to 84, wherein the PPARα agonist is GW7647, PEA, or OEA.

P Embodiment 91. A method of decreasing pain hypersensitivity in a subject after a traumatic pain event, the method comprising administering an effective amount of a composition to the subject, wherein the composition comprises at least one purified free amino acid, at least one purified fatty acid, and a purified fatty acid amide, wherein pain hypersensitivity is decreased in the subject.

P Embodiment 92. The method of embodiment 91, wherein the fatty acid amide is purified palmitoylethanolamide (PEA).

P Embodiment 93. The method of embodiment 91, wherein the pain hypersensitivity is induced by acute/inflammatory pain.

P Embodiment 94. The method of embodiment 91, wherein the inflammatory pain is IL-6 mediated.

P Embodiment 95. A composition comprising at least one purified free amino acid, at least one purified fatty acid, and purified fatty acid amide.

P Embodiment 96. The composition of embodiment 95 wherein the fatty acid amide is purified palmitoylethanolamide (PEA).

P Embodiment 97. The composition of embodiment 95 or embodiment 96, further comprising an artificial sweetener.

P Embodiment 98. A dosage form comprising the composition of any one of embodiments 95 to 97, wherein the dosage form is a powder.

P Embodiment 99. The dosage form of embodiment 98, wherein the dosage form is about 100 gr per dose.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A method of preventing chronic pain in a subject after a traumatic pain event, preventing chronic pain in a subject with diabetes, or preventing peripheral neuropathic pain in a subject previously treated with an anti-cancer agent the method comprising administering an effective amount of a composition to the subject, wherein the composition comprises at least one purified free amino acid, at least one purified fatty acid, and purified fatty acid amide wherein the pain is prevented in the subject.

2. The method of claim 1, wherein the fatty acid amide is purified palmitoylethanolamide (PEA).

3. The method of claim 1, wherein the composition is administered orally.

4. The method of claim 1, wherein the purified fatty acid is oleic acid or erucic acid.

5. The method of claim 4, wherein the subject is a human and the dosage range of oleic acid is between 48 to 496 mg/kg.

6. The method of claim 4, wherein the subject is a human and the dosage range of erucic acid is 2 to 25 mg/kg.

7. The method of claim 1, wherein the purified free amino acid is alanine, threonine, proline, serine, leucine, isoleucine, valine, phenylalanine, tyrosine, methionine, cysteine, or tryptophan.

8.-20. (canceled)

21. The method of claim 1, wherein the composition is administered between about 0 days and about 30 days after the traumatic pain event.

22. The method of claim 1, wherein the composition is administered between about 30 days and about 1 day before the traumatic pain event.

23. The method of claim 1, wherein the traumatic pain event is due to physical injury, invasive surgery, or acute illness.

24. The method of claim 1, wherein the physical injury is accidental physical injury.

25.-26. (canceled)

27. The method of claim 23, wherein the invasive surgery is knee arthroplasty, hip replacement surgery, mastectomy, open-heart surgery, hernia repair, thoracotomy, caesarian section, amputation, or open cholecystoctomy.

28. The method of claim 27, wherein the composition is administered perioperatively.

29. The method of claim 1, further comprising administering an agent between about 1 day and about 30 days after the traumatic pain event and wherein the agent is a NAAA inhibitor, FAAH inhibitor, PPARα agonist, PEA, acetyl-L-carnitine, α-lipoic acid, or olesoxime.

30.-80. (canceled)

81. The method of claim 1, further comprising administering an effective amount of an agent to the subject, wherein said agent is administered between about 1 day and about 30 days after a traumatic pain event and wherein said agent is a NAAA inhibitor, FAAH inhibitor, PPARα agonist, acetyl-L-carnitine, α-lipoic acid, or olesoxime.

82.-83. (canceled)

84. The method of claim 81, wherein the agent is a NAAA inhibitor, a FAAH inhibitor, or a PPARα agonist.

85.-86. (canceled)

87. The method of claim 81, wherein the FAAH inhibitor is URB937 or an analog of URB 937.

88.-94. (canceled)

95. A composition comprising at least one purified fatty acid, and a purified fatty acid amide, wherein the fatty acid amide is purified palmitoylethanolamide (PEA).

96.-97. (canceled)

98. A dosage form comprising the composition of claim 95, wherein the dosage form is a powder.

99. The dosage form of claim 98, wherein the dosage form is about 100 gr per dose.
